(19) United States
(12) Patent Application Publication
(10) Pub. No.: US 2023/0102840 A1
(43) Pub. Date: Mar. 30, 2023

(54) COMPOSITIONS AND METHODS RELATING TO COMBINATORIAL HYALURONIC ACID CONJUGATES

(71) Applicant: PRESIDENT AND FELLOWS OF HARVARD COLLEGE, Cambridge, MA (US)

(72) Inventors: Samir MITRAGOTRI, Cambridge, MA (US); Marwa Ahmed SALLAM, Cambridge, MA (US)

(73) Assignee: PRESIDENT AND FELLOWS OF HARVARD COLLEGE, Cambridge, MA (US)

(21) Appl. No.: 17/921,484

(22) PCT Filed: Jun. 21, 2021
(86) PCT No.: PCT/US2021/038203

§ 371 Date: Oct. 26, 2022

Related U.S. Application Data
(60) Provisional application No. 63/042,652, filed on Jun. 23, 2020.

(51) Int. Cl. A61K 47/61; A61P 35/04

(52) U.S. Cl. CPC A61K 47/61; A61P 35/04

(57) ABSTRACT

Described herein are compositions comprising polymers conjugated to at least one immunomodulator, e.g., a) a retinoid and an imidazoquinoline, b) a retinoid and a HDAC inhibitor, or c) a HDAC inhibitor and an imidazoquinoline. Further described herein are methods of using said compositions, e.g., in modulating immune responses and/or treating a disease such as cancer.

### CROSS-REFERENCE TO RELATED APPLICATIONS

This application claims benefit under 35 U.S.C. § 119(e) of U.S. Provisional Application No. 63/042,652 filed Jun. 23, 2020, the contents of which are incorporated herein by reference in their entirety.

### TECHNICAL FIELD

The technology described herein relates to cancer therapeutics, e.g., combinatorial therapies.

### BACKGROUND

Existing cancer therapeutics are limited by a number of disadvantages, such as low bioavailbility, low solubility, systemic toxicity, and low tissue and/or cell permeability. Improvements on these existing therapeutics, which improve their delivery and reduce their side effects are one route to improve cancer therapy. Such improvements can provide better clinical outcomes, reduce the duration of treatment, and avoid relapse or the development of drug resistance.

### SUMMARY

The inventors have found that by conjugating certain drugs to hyaluronic acid, they can improve cell and tissue delivery, lower the unwanted systemic spread of drugs beyond therapeutic areas, improve efficacy, and provide synergy not anticipated by the performance of the free drugs. The inventors have further demonstrated that these new conjugates can exert at least part of their therapeutic effect by inducing anti-cancer phenotypes in macrophages.

In one aspect of any of the embodiments, described herein is a composition comprising a polymer conjugated to at least one immunomodulator. In some embodiments of any of the aspects, the polymer is hyaluronic acid.

In some embodiments of any of the aspects, the immunomodulator is a retinoid, a HDAC inhibitor, or an imidazoquinoline. In some embodiments of any of the aspects, the immunomodulator is selected from the group consisting of: bexarotene, vorinostat, or resiquimod. In some embodiments of any of the aspects, the composition comprises at least two immunomodulators. In some embodiments of any of the aspects, the composition comprises at least a) a retinoid and an imidazoquinoline, b) a retinoid and a HDAC inhibitor, or c) a HDAC inhibitor and an imidazoquinoline. In some embodiments of any of the aspects, the composition comprises at least a) a retinoid and an imidazoquinoline or b) a retinoid and a HDAC inhibitor. In some embodiments of any of the aspects, the composition comprises resiquimod and bexarotene. In some embodiments of any of the aspects, the composition comprises vorinostat and bexarotene.

In some embodiments of any of the aspects, the at least two immunomodulators are conjugated to the same polymer molecule such that each polymer molecule comprises at least one molecule of each of the at least two immunomodulators. In some embodiments of any of the aspects, the at least two immunomodulators are each conjugated to separate polymer molecules such that each polymer molecule comprises only one of the at least two immunomodulators. In some embodiments of any of the aspects, the at least two immunomodulators are present in equimolar amounts. In some embodiments of any of the aspects, the at least two immunomodulators are present in a ratio of from 1:2 to 2:1. In some embodiments of any of the aspects, the at least two immunomodulators are present in a ratio of from 1:1 to 2:1 of bexarotene:a second immunomodulator.

In some embodiments of any of the aspects, the at least one immunomodulator is conjugated directly to the polymer. In some embodiments of any of the aspects, the conjugation is via an ester bond and/or via an amide bond.

In some embodiments of any of the aspects, each polymer molecule is about 5 kDa to about 250 kDa in size. In some embodiments of any of the aspects, each polymer molecule is about 50 kDa in size.

In some embodiments of any of the aspects, the at least one immunomodulator is present at a concentration of about 2.0 to about 25.0 mol %. In some embodiments of any of the aspects, the at least one immunomodulator is present at a concentration of about 4.0 to about 10.0 mol %.

In some embodiments of any of the aspects, the polymer is hyaluronic acid and the composition does not comprise another polymer.

In one aspect of any of the embodiments, described herein is a cellular composition comprising a composition as described herein, a) adhered to or located on the surface of a macrophage or monocyte, or b) present in a macrophage or monocyte. In some embodiments of any of the aspects, the macrophage or monocyte is obtained from a subject. In some embodiments of any of the aspects, the macrophage or monocyte is autologous to a subject.

In one aspect of any of the embodiments, described herein is a pharmaceutical composition comprising the composition or cellular composition described herein and optionally a pharmaceutically acceptable carrier.

In one aspect of any of the embodiments, described herein is a method of increasing the activity of level of a M1 macrophage phenotype and/or decreasing the activity or level of a M2 macrophage phenotype, the method comprising contacting at least one macrophage with a composition described herein. In one aspect of any of the embodiments, described herein is a method of stimulating an immune response in a subject in need thereof, the method comprising administering a composition as described herein to the subject. In some embodiments of any of the aspects, the subject is in need of treatment for cancer. In some embodiments of any of the aspects, the cancer is melanoma. In some embodiments of any of the aspects, the cancer is metastatic.

In some embodiments of any of the aspects, the composition is administered by intravenous or intratumoral injection. In some embodiments of any of the aspects, the composition comprises resiquimod and bexarotene and is administered by intravenous or intratumoral injection. In some embodiments of any of the aspects, the composition is administered topically. In some embodiments of any of the aspects, the composition comprises vorinostat and bexarotene and is administered topically.

### BRIEF DESCRIPTION OF THE DRAWINGS

FIGS. 1A-1C depict the synthesis of the HA-RES and HA-BEX conjugates of Example 1.

FIGS. 2A-2C depict the characterization of HA-RES (right) and HA-BEX (left) and HA-RES-BEX dual conjugates. (FIG. 2A) NMR conjugation efficiency (4.5-5 molar % for HA-RES, 8-9 molar % for HA-BEX in the single conjugate 5.5-6 molar % in the dual conjugate) (FIG. 2B) Hydrodynamic diameter by (DLS) 122.4, 138.8 and 213.4 nm for HA-RES, HA-BEX and Dual conjugate respectively and (FIG. 2C) TEM Scale bar 500 nm).

FIGS. 3A-3D depict the effect of RES and HA-RES on the viability of BMDM (FIG. 3A) and production of cytokines (FIGS. 3B, 3C) and uptake of HA-RES by BMDM (FIG. 3D).

FIGS. 4A-4B depict the effect of BEX and HA-BEX at different concentrations on the viability of BMDM (FIG. 4A) and on the production of CCL22 in the supernatant of BMDM (FIG. 4B).

FIGS. 5A-5D depict the effect of combining HA-RES and HA-BEX at selected ratios on the viability of macrophages (FIG. 5A) and on the production of IL-6 (FIG. 5B), TNF-alpha (FIG. 5C) and CCL2 (FIG. 5D).

FIGS. 6A-6C depict phenotypic evaluation of BMDM after treatment with different concentration with RES and HA-RES (FIG. 6A), BEX and HA-BEX (FIG. 6B) and a combination of HA-RES and HA-BES (FIG. 6C) at selected ratios.

FIGS. 7A-7C depict the effect of HA-BEX, HA-RES and their combination on the viability of B16F10-Luc melanoma cells cocultured with BMDM.

FIGS. 8A-8C depict the uptake of HA-RES and dual conjugate by BMDM (FIG. 8A) Quantitative uptake of the conjugates by BMDM (FIG. 8B) Flow cytometry histogram showing the uptake of HA-BEX-RES-Alexa488 by BMDM with and without pretreatment of the cells with HA. Black line: uptake of the florescent conjugate applied 2 hr after pretreatment of the cells with HA. Pink line: uptake of the conjugate without HA pretreatment (Higher florescence intensity). (FIG. 8C) confocal microscopy showing the localization of the dual conjugate within the lysosomal compartment of the BMDM.

FIGS. 9A-9B depict a phagocytosis study (FIG. 9A) schematic representation of the experiment. BMDM were stained by Vivotrack 680 and plated into non-TC treated 12 well plates (2.5×10.5 cells/well) overnight. The cells were then incubated with the different treatments including HA-RES, HA-BEX and the dual conjugate each at concentrations equivalent to 25 μM of each drug, for 48 hr. B16F10 melanoma cells were labelled with cell trace violet and incubated (1×105 cells/well) with the stained treated BMDM at a ratio of 1:2.5. After 4 hr, the supernatant was aspirated the cells were dislodged then suspended in flow cytometry buffer for flow cytometry analysis. Percentage of phagocytosis was determined by the percentage of violet positive cells within red stained BMDM cell gate. (FIG. 9B) A significant increase in the % phagocytosis was observed in the cells treated with the dual conjugate.

FIGS. 10A-10D depict cell morphology of BMDM after treatment with HA-RES, HA-BEX and dual conjugate. Following treatment with HA-RES, cell morphology shifted from elongated structures observed in untreated BMDMS (FIG. 10A) to round, and flattened structures (FIG. 10B), characteristic of M1 activated macrophages. BMDM treated with a Dual HA-BEX/HA-RES showed mostly round morphology (FIG. 10D).

FIGS. 11A-11B depict a biodistribution study (FIG. 11A) representative images of the organs collected after 4 and 8 hr and imaged by IVIS. (FIG. 11B) Quantitative biodistribution of HA-RES-BEX-Alexa 647. The amount detected in the tumor after 4 h and 8 hr was 43.53±9.37 and 51.65±7.15 ug/g of tissue that correspond to approx. 2.17 and 2.51 ug of the conjugated drugs. A substantial amount of the conjugate also is localized in the spleen, which is an important target for systemic immune response.

FIGS. 12A-12E depict the efficacy of HA-RES, HA-BEX and dual conjugate in B16F10-Luc model. (FIG. 12A) Schematic chart of the treatment schedule. (FIG. 12B) Bioluminescence images of subcutaneous melanoma at different time points after inoculation. (FIG. 12C) Graph shows the tumor growth profiles (FIG. 12D) melanoma tumors excised from the mice at the time of study termination. (FIG. 12E) Mice weight was monitored to reflect the general well-being of the animals. No mouse lost more than 15% body weight in the period of the study.

FIGS. 13A-13B depict tumor-associated immune cell phenotyping. FIG. 13A. All the treatment groups showed an increase in the expression of iNOS compared to the control. The effect was highly significant for the group that received the dual conjugate. In addition a significant reduction in CD206 was observed for all the treatment groups. Over all, the group treated with the dual conjugate showed a significant increase in the M1/M2 ratio. (FIG. 13B) However, for the surface markers CD80+M1 macrophages showed no significant differences across all the groups (not shown). Groups receiving HA-RES and dual conjugate showed a significant reduction in the VEGF (surface M2 marker). For the surface M1/M2 ratio, the group treated with the dual conjugate showed a significant increase.

FIGS. 14A-14B depict the scheme for chemical synthesis of (FIG. 14A) HA-BEX and (FIG. 14B)HA-VOR

FIGS. 15A-15F depict TEM showing spherical structures of (FIG. 15A)HA-BEX (FIG. 15B) HA-VOR; DLS showing size distribution of (FIG. 15C) HA-BEX and (FIG. 15D) HA-VOR and zeta potential of (FIG. 15E) HA-BEX and (FIG. 15F) HA-VOR.

FIGS. 16A-16D depict in vitro cytotoxicity on A431 cells comparing the activity of (FIG. 16A)BEX and HA-BEX, (FIG. 16B) VOR and HA-VOR. Data represents Mean±SD (n=6). (FIG. 16C) combination index of BEX and VOR at different ratios showing synergy between the two drugs at ratio of 2:1 and (FIG. 16D) effect of the combined conjugates at a ratio of 2:1 compared to the individual conjugates.

FIGS. 17A-17B. (FIG. 17A) In vitro cytotoxicity on HUT 78 cells comparing the activity of the combined conjugates at a ratio of 2:1 to the individual conjugates (FIG. 17B) IC50 values of HA-BEX and HA-VOR and their combination showing reduction in the IC50 of each conjugate in the combination confirming the synergy at this ratio.

FIG. 18 depicts uptake of HA-VOR-Alexa488 and HA-BEX Alexa 647 by A431 cells. Flow cytometry histogram plots showing increase in the percentage of positive cells and in florescent intensity by time, CLSM showing the uptake of after 4 h.

FIGS. 19A-19B depict the effect of BEX, VOR, and their HA conjugates on the viability of (FIG. 19A) primary human fibroblasts and (FIG. 19B) Primary human keratinocytes.

FIGS. 20A-20B depict CLSM images of Human skin explant after application of (FIG. 20A), HA-VOR-Alexa 488 (green) and HA-BEX-Alexa 647 (red) For all (I) represents skin back ground, (II) the conjugate fluorophore channel, (III) merged images (FIG. 20B) both conjugates applied together (I) skin stained with DAPI, (II) Alexa 488 channel (HA-VOR), (III) Alexa 647 channel (HA-BEX), (IV) merged images showing all the channels and (V) plot of florescence intensity of both conjugates versus skin depth.

FIG. 21A. Quantification of florescence intensity across the skin depth by Zen 3.1 (n=3) after permeation studies. FIG. 21B. RP-HPLC quantification of BEX and VOR extracted from the skin layers after the topical application of HA-BEX and HA-VOR.

FIGS. 22A-22C depict the ¹H NMR spectra of: (FIG. 22A) HA-BEX, (FIG. 22B) Bexarotene, and (FIG. 22C) Hyaluronic acid.

FIGS. 23A-23C depict the ¹H NMR spectra of (FIG. 23A) HA-VOR, (FIG. 23B) Vorinostat, (FIG. 23C) Hyaluronic acid.

FIGS. 24A-24D depict the uptake of HA-VOR-Alexa488 and HA-BEX Alexa 647 by HUT 78 cells: (FIGS. 24A & 24C) flow cytometry histogram plots showing increase in the percentage of positive cells and in florescent intensity by time, (FIGS. 24B & 24D) CLSM showing the uptake of after 4 h.

### DETAILED DESCRIPTION

The inventors have found that combinations of immunomodulators display increased efficacy when conjugated to a polymer (e.g., hyaluronic acid). These conjugates display increased delivery kinetics, improved immunomodulation, and improved synergy. Without wishing to be bound by theory, the improved synergy may be due to the improved control of relative doses. Accordingly, in one aspect of any of the embodiments, provided herein is a composition comprising a polymer (e.g., hyaluronic acid) conjugated to at least one immunomodulator.

In some embodiments of any of the aspects, the polymer is a hydrophilic polymer. In some embodiments of any of the aspects, the polymer is a water-soluble polymer. In some embodiments of any of the aspects, the polymer is a water-soluble hydrophilic polymer. In some embodiments of any of the aspects, the polymer is a biodegradable polymer. In some embodiments of any of the aspects, the polymer is a biodegradable, water-soluble, hydrophilic polymer.

In some embodiments of any of the aspects, the polymer can comprise a functional group that permits chemical conjugation. Functional groups that permit chemical conjugation can include, by way of non-limiting example, a carboxyl, an amine, and an alcohol. In some embodiments of any of the aspects, the polymer can comprise a carboxyl, an amine, and/or an alcohol functional group.

In some embodiments of any of the aspects, the polymer is a biocompatible polymer. In some embodiments of any of the aspects, the polymer is a glycosaminoglycan. In some embodiments of any of the aspects, the polymer is a nonsulfated polymer. In some embodiments of any of the aspects, the polymer is a polymer that permits chemical conjugation.

Examples of suitable polymers, e.g., of the foregoing types of polymers are known in the art and include, by way of non-limiting example synthetic polypeptides (e.g., polygutamic acid); polysaccharides (e.g., chitosan and dextran); and synthetic polymers (e.g., polyvinyl alcohols). Further examples are known in the art and described, e.g., in Babak Ghanbarzadeh and Hadi Almasi (Jun. 14, 2013). Biodegradable Polymers, Biodegradation—Life of Science, Rolando Chamy and Francisca Rosenkranz, IntechOpen, DOI: 10.5772/56230; Finch C. A. (1987) Hydrophilic polymers. In: Dyson R. W. (eds) Specialty Polymers. Springer, Boston, Mass. doi.org/10.1007/978-1-4615-7894-9_5; Schmidt. Polymers (Basel) 2019 11:693; and Traubel H. (1999) Hydrophilic Polymers. In: New Materials Permeable to Water Vapor. Springer, Berlin, Heidelberg; each of which is incorporated by reference herein in its entirety. In some embodiments of any of the aspects, the polymer is hyaluronic acid; polygutamic acid; chitosan; dextran; and/or a polyvinyl alcohol. In some embodiments of any of the aspects, the polymer is hyaluronic acid.

Hyaluronic acid (HA) or hyaluronan is a nonsulfated glycosaminoglycan comprising a polymer of D-glucuronic acid and N-acetyl-D-glucosamine disaccharides joined by β-(1→4) and β-(1→3) glycosidic bonds. Hyaluronic acid polymers can comprise up to about 25,000 disaccharide units (repeats) in a single hyaluronic molecule, and hyaluronic acid molecules typically range in size from about 5,000 Da to about 20,000,000 Da. In some embodiments of any of the aspects, the composition does not comprise a polymer other than hyaluronic acid. In some embodiments of any of the aspects, the composition does not comprise a glycosaminoglycan other than hyaluronic acid.

In some embodiments of any of the aspects, a composition described herein comprises a polymer (e.g., hyaluronic acid) that comprises, consists of, or consists essentially of a polymer (e.g., hyaluronic acid) molecules having a molecular weight of about 1 kDa to about 500 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of 1 kDa to 500 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of about 5 kDa to about 250 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of 5 kDa to 250 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of about 10 kDa to about 100 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of 10 kDa to 100 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of about 40 kDa to about 60 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of 40 kDa to 60 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of about 50 kDa. In some embodiments of any of the aspects, a composition described herein comprises a polymer (e.g., hyaluronic acid) that comprises, consists of, or consists essentially of a polymer (e.g., hyaluronic acid) molecules having a molecular weight of 50 kDa.

As used herein, “immunomodulator” refers to refers to an agent with the capacity to modify the immune system of a subject, for example, the agent can induce, amplify, attenuate, or prevent an immune response. Preferably, the agent induces or amplifies the immune response and/or polarizes macrophages to an M1 phenotype. It should be understood that while any antigen can be an immunomodulator in the sense that it induces an immune response, an “immunomodulator” as the term is used herein modifies the immune microenvironment of a macrophage or tumor, e.g., in terms of recruitment of immune effectors, activity of immune effectors, or suppression of immunosuppressive factors, their expression, or their activity. Exemplary immunomodulators are retinoids, HDAC inhibitors, and imidazoquinolines.

In some embodiments of any of the aspects, the composition comprises at least two immunomodulators.

In some embodiments of any of the aspects, the composition comprises at least a retinoid and a HDAC inhibitor. In some embodiments of any of the aspects, the composition comprises at least a retinoid and imidazoquinoline. In some embodiments of any of the aspects, the composition comprises at least an imidazoquinoline and a HDAC inhibitor. In some embodiments of any of the aspects, the composition comprises at least a retinoid, an imidazoquinoline, and a HDAC inhibitor. In some embodiments of any of the aspects, the composition comprises at least one of: at least one retinoid, at least one imidazoquinoline, and at least one HDAC inhibitor. In some embodiments of any of the aspects, the composition comprises at least two of: at least one retinoid, at least one imidazoquinoline, and at least one HDAC inhibitor. In some embodiments of any of the aspects, the composition comprises at least one retinoid, at least one imidazoquinoline, and at least one HDAC inhibitor. In some embodiments of any of the aspects, the composition can comprise two or more species of any of the foregoing genuses of immunomodulators, e.g., two or more retinoids, two or more HDAC inhibitors, two or more imidazoquinolines, or any combination thereof. In some embodiments of any of the aspects, the composition described herein does not comprise an imidazoquinoline. In some embodiments of any of the aspects, the composition described herein does not comprise a retinoid. In some embodiments of any of the aspects, the composition described herein does not comprise an HDAC inhibitor.

As used herein, “retinoid” refers to vitamers of vitamin A and chemically-related compounds comprising a cyclic end group, a polyene side chain, and a polar end group. Retinoids are well known in the art and include retinol, retinal, tretinoin, isotretinoin, alitretinoin, etretinate, acetretin, adapalene, tazarotene, and bexarotene. In some embodiments of any of the aspects, the at least one retinoid is retinol, retinal, tretinoin, isotretinoin, alitretinoin, etretinate, acetretin, adapalene, tazarotene, and/or bexarotene. In some embodiments of any of the aspects, the at least one retinoid is adapalene, tazarotene, and/or bexarotene. In some embodiments of any of the aspects, the at least one retinoid is alitretinoin, tretinoin, and/or bexarotene. In some embodiments of any of the aspects, the at least one retinoid comprises, consists essentially of, or consists of bexarotene.

As used herein, “HDAC inhibitor” refers to an inhibitor of a histone deacetylase (HDAC). As used herein, the term “inhibitor” refers to any compound, natural or synthetic, which can reduce the activity of a target protein or signaling pathway. An inhibitor can be, for example, a peptide, a protein, an antibody, a peptidomimetic, an amino acid, an amino acid analog, a polynucleotide, a polynucleotide analog, an aptamer, a nucleotide, a nucleotide analog, an organic or inorganic compound. An inhibitor may attenuate or prevent the activity of a target protein either directly or indirectly. Direct inhibition can be obtained, for instance, by binding to a protein and preventing the protein from interacting with an endogenous molecule, such as an enzyme, a substrate, or other binding partner, thereby diminishing the activity of the protein. For instance, an inhibitor may bind an enzyme active site and sterically preclude binding of an endogenous substrate at this location, thus decreasing the enzymatic activity of the protein. Alternatively, indirect inhibition can be obtained, for instance, by binding to a protein that promotes the activity of a target protein by inducing a conformational change or catalyzing a chemical modification of the target protein. For instance, indirect inhibition of a target protein may be achieved by binding and inactivating a kinase that catalyzes the phosphorylation of, and thus activates, the target protein.

As used herein, the terms “histone deacetylase” and “HDAC” refer to any one of a family of enzymes that catalyze the removal of acetyl groups from the F-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

HDAC inhibitors are known in the art and can include hydroxamic acids (e.g., trichostatin A, panobinsostat (LBH589), belinostat (PXD101), dacinostate (LAW824), Scriptaid, vorinostat (SAHA), pracinostat (SB939)); cyclic tetrapeptides and depsipeptides (e.g., trapoxin B, romidepsin (istodax)); benzamides (e.g., entinostat (MS-275), tacedinaline (C1994), and mocetinostate (MGCD0103)); electrophilic ketones; aliphatic acid compounds (e.g., phenylbutyrate and valproic acid); butyrylhydroxamic acid; M344; AR-42; CUDC-101, sodium phenylbutyrate; tasquinimod; quisinostat (JNJ-26481585); CUDC-907; tubastatin A HCl; PCI-34051; droxinostat; PCI-24781 (Abexinostat); RGFP966; Rocilinostat (ACY-1215); RG2833 (RGFP109); resminostat; BRD73954; BG45; 4SC-202; CAY10603; LMK-235; nexturastat A; TMP269; HPOB; cambinol; and anacardic acid. In some embodiments of any of the aspects, the at least one HDAC inhibitor can be selected from trichostatin A, panobinsostat (LBH589), belinostat (PXD101), dacinostate (LAW824), Scriptaid, vornostat (SAHA), and pracinostat (SB939). In some embodiments of any of the aspects, the at least one HDAC inhibitor comprises, consists essentially of, or consists of vorinostat (SAHA).

As used herein “imidazoquinoline” refers to a compound comprising an imidazoquinoline structure. In some embodiments of any of the aspects, the imidazoquinoline can be an imidazoquinoline having TLR agonist activity. Imidazoquionlines known in the art to comprise TLR agonist activity include imiquimod, resiquimod, gardiquimod, R-848; 3M-002 (CL075); 3M-013; 3M003 (4-amino-2-(ethoxymetiyl)-α-dimethyl-6,7,8,9-tetrahydro-1H-imidazo[4,5-c]quinoline-1-ethanolhydrate); 852; S-34240; 854A; and CL097. In some embodiments of any of the aspects, the at least one imidazoquinoline is selected from imiquimod, resiquimod, and gardiquimod. In some embodiments of any of the aspects, the at least one imidazoquinoline comprises, consists essentially of, or consists of resiquimod, imiquimod, and/or gardiquimod. In some embodiments of any of the aspects, the at least one imidazoquinoline comprises, consists essentially of, or consists of resiquimod.

In some embodiments of any of the aspects, the immunomodulators of a composition described herein comprise resiquimod and bexarotene. In some embodiments of any of the aspects, the immunomodulators of a composition described herein consist essentially of resiquimod and bexarotene. In some embodiments of any of the aspects, the immunomodulators of a composition described herein consist of resiquimod and bexarotene. In some embodiments of any of the aspects, the composition described herein comprises a polymer (e.g., hyaluronic acid), resiquimod, and bexarotene. In some embodiments of any of the aspects, the composition described herein consists essentially of a polymer (e.g., hyaluronic acid), resiquimod, and bexarotene. In some embodiments of any of the aspects, the composition described herein consists of a polymer (e.g., hyaluronic acid), resiquimod, and bexarotene.

In some embodiments of any of the aspects, the immunomodulators of a composition described herein comprise vorinostat and bexarotene. In some embodiments of any of the aspects, the immunomodulators of a composition described herein consist essentially of vorinostat and bexarotene. In some embodiments of any of the aspects, the immunomodulators of a composition described herein consist of vorinostat and bexarotene. In some embodiments of any of the aspects, the composition described herein comprises a polymer (e.g., hyaluronic acid), vorinostat, and bexarotene. In some embodiments of any of the aspects, the composition described herein consists essentially of a polymer (e.g., hyaluronic acid), vorinostat, and bexarotene. In some embodiments of any of the aspects, the composition described herein consists of a polymer (e.g., hyaluronic acid), vorinostat, and bexarotene.

In some embodiments of any of the aspects, the immunomodulators of a composition described herein comprise at least two immunomodulators selected from resiquimod, vorinostat and bexarotene. In some embodiments of any of the aspects, the immunomodulators of a composition described herein consist essentially of at least two immunomodulators selected from resiquimod, vorinostat and bexarotene. In some embodiments of any of the aspects, the immunomodulators of a composition described herein consist of at least two immunomodulators selected from resiquimod, vorinostat and bexarotene. In some embodiments of any of the aspects, the composition described herein comprises a polymer (e.g., hyaluronic acid) and at least two immunomodulators selected from resiquimod, vorinostat and bexarotene. In some embodiments of any of the aspects, the composition described herein consists essentially of a polymer (e.g., hyaluronic acid) and at least two immunomodulators selected from resiquimod, vorinostat and bexarotene. In some embodiments of any of the aspects, the composition described herein consists of a polymer (e.g., hyaluronic acid) and at least two immunomodulators selected from resiquimod, vorinostat and bexarotene.

The term “conjugate,” as it relates to polymer-immunomodulator conjugate refers to two or more molecular structures that are linked by a direct or indirect covalent or non-covalent bond. Non-covalent interactions include, but are not limited to, electrostatic interactions, hydrogen bonding interactions, van der Waals interactions, dipole-dipole interactions, π-π stacking, magnetic interactions, and metal coordination. Preferably, the conjugation is via covalent bonds.

The immumoodulators are conjugated directly or indirectly, via linkers, to the backbone or side chains of one or more polymers (e.g., hyaluronic acid). In a polymer-immunomodulator conjugate comprising two or more immunomodulators, the strengths of the bonds involved in direct conjugation, and the strengths of the bonds in the linkers, may be the same, different, or a combination thereof (i.e. some of the bond strengths are the same while others are different). In some embodiments of any of the aspects, the side chains have the same or different chemical moieties. The bond strengths of the bonds, directly conjugating the two or more immunomodulators to the polymers are the same, different, or a combination thereof (i.e. some of the bond strengths are the same while others are different). The bond strengths of the bonds indirectly conjugating the two or more immunomodulators via linkers are the same, different, or a combination thereof (i.e. some of the bond strengths are the same while others are different). For example, when the bonds or linkers have the same bond strength, each of the immunomodulators is connected to the polymer via the same bond or the same linker. When the bonds or linkers have different bond strengths, each of the immunomodulators is connected to the polymer via a different bond or different linker.

The linkers (or a portion thereof) or one or more of the bonds between a drug and a linker may be cleaved at the same rate or a different rate as the cleavage of another linker or one or more of the bonds between a different immunomodulator and another linker in the pharmaceutical composition. Cleavage can occur by any suitable mechanism, such as via hydrolysis, enzymatic cleavage, the application of thermal energy, photoenergy, or a combination thereof.

The linkers may be homo-bifunctional or hetero-bifuctional. In some instances, combinations of homo-bifunctional linkers and hetero-bifunctional linkers are used. Examples of homo-bifunctional linkers include, but are not limited to adipic acid dihydrazide, amino acids such as glycine, aldehydes such as ethanedial, pyruvaldehyde, 2-formyl-malonaldehyde, glutaraldehyde, adipaldehyde, heptanedial, octanedial; di-glycidyl ether, diols such as 1,2-ethanediol, 1,3-propanediol, 1,4-butanediol, 2,3-butanediol, 1,5-pentanediol, benzene-1,4-diol, 1,6-hexanediol, tetra(ethylene glycol) diol), PEG, di-thiols such as 1,2-ethanedithiol, 1,3-propanedithiol, 1,4-butanedithiol, 2,3-butanedithiol, 1,5-pentanedithiol, benzene-1,4-dithiol, 1,6-hexanedithiol, tetra(ethylene glycol) dithiol), di-amine such as ethylene diamine, propane-1,2-diamine, propane-1,3-diamine, N-methylethylenediamine, N,N′-dimethylethylenediamine, pentane-1,5-diamine, hexane-1,6-diamine, spermine and spermidine, divinyladipate, divinylsebacate, diamine-terminated PEG, double-ester PEG-N-hydroxysuccinimide, and di-isocyanate-terminated PEG. In a preferred embodiment, the homo-bifunctional linker is adipic acid dihydrazide.

Examples of hetero-bifunctional linkers include, but are not limited to, epichlorohydrin, S-acetylthioglycolic acid N-hydroxysuccinimide ester, 5-azido-2-nitrobenzoic acid N-hydroxysuccinimide ester, 4-azidophenacyl bromide, bromoacetic acid N-hydroxysuccinimide ester, N-(3-dimethylaminopropyl)-N′-ethylcarbodiimide, Iodoacetic acid N-hydroxysuccinimide ester, 4-(N-mMaleimido)benzophenone 3-(2-pyridyldithio)propionic acid N-hydroxysuccinimide ester 3-maleimidobenzoic acid N-hydroxysuccinimide ester, N,N′-cystamine-bis-acrylamide, N,N′-methylene-bis-acrylamide and N,N′-ethylene-bis-acrylamide.

In some embodiments of any of the aspects, the conjugation is direct. In some embodiments of any of the aspects, the conjugation is via an amide or ester bond. In some embodiments of any of the aspects, the conjugation of a first immunomodulator is via an amide bond and the conjugation of the second immunomodulator is via an ester bond.

Where a composition comprises two or more immunomodulators, the immunomodulators can be conjugated to the a polymer (e.g., hyaluronic acid) either: 1) such that each individual a polymer (e.g., hyaluronic acid) molecule is conjugated to only one species of immunomodulatory; 2) such that each individual a polymer (e.g., hyaluronic acid) molecule is conjugated to at least one molecule of each of at least two species of immunomodulators; 3) such that each individual a polymer (e.g., hyaluronic acid) molecule is conjugated to at least one molecule of each of the immunomodulator species present in the composition; or 4) the composition can comprise mixtures of any of the foregoing arrangements.

In some embodiments of any of the aspects, a composition described herein can further comprise a polymer (e.g., hyaluronic acid) molecules not conjugated to any immunomodulator. In some embodiments of any of the aspects, every a polymer (e.g., hyaluronic acid) molecule in a composition described herein is conjugated to at least one immunomodulator molecule.

In some embodiments of any of the aspects, the at least two immunomodulators are present in the composition at equimolar amounts. In some embodiments of any of the aspects, the at least two immunomodulators are present in the composition at a molar ratio of from about 1:4 to about 4:1. In some embodiments of any of the aspects, the at least two immunomodulators are present in the composition at a molar ratio of from 1:4 to 4:1. In some embodiments of any of the aspects, the at least two immunomodulators are present in the composition at a molar ratio of from about 1:2 to about 2:1. In some embodiments of any of the aspects, the at least two immunomodulators are present in the composition at a molar ratio of from 1:2 to 2:1.

In some embodiments of any of the aspects, the at least two immunomodulators are conjugated to the same a polymer (e.g., hyaluronic acid) molecule at a molar ratio of from about 1:4 to about 4:1. In some embodiments of any of the aspects, the at least two immunomodulators are conjugated to the same a polymer (e.g., hyaluronic acid) molecule at a molar ratio of from 1:4 to 4:1. In some embodiments of any of the aspects, the at least two immunomodulators are conjugated to the same a polymer (e.g., hyaluronic acid) molecule at a molar ratio of from about 1:2 to about 2:1. In some embodiments of any of the aspects, the at least two immunomodulators are conjugated to the same a polymer (e.g., hyaluronic acid) molecule at a molar ratio of from 1:2 to 2:1.

In some embodiments of any of the aspects, the at least two immunomodulators are bexarotene and a second immunomodulator and are present in the composition at a molar ratio of from about 1:1 to about 4:1 of bexarotene:the second immunomodulator (e.g., vorinostat or resiquimod). In some embodiments of any of the aspects, the at least two immunomodulators are bexarotene and a second immunomodulator and are present in the composition at a molar ratio of from 1:1 to 4:1 of bexarotene:the second immunomodulator (e.g., vorinostat or resiquimod). In some embodiments of any of the aspects, the at least two immunomodulators are bexarotene and a second immunomodulator and are present in the composition at a molar ratio of from about 1:1 to about 2:1 of bexarotene:the second immunomodulator (e.g., vorinostat or resiquimod). In some embodiments of any of the aspects, the at least two immunomodulators are bexarotene and a second immunomodulator and are present in the composition at a molar ratio of from 1:1 to 2:1 of bexarotene:the second immunomodulator (e.g., vorinostat or resiquimod).

In some embodiments of any of the aspects, the at least two immunomodulators are bexarotene and a second immunomodulator and are conjugated to the same a polymer (e.g., hyaluronic acid) molecule at a molar ratio of from about 1:1 to about 4:1 of bexarotene:the second immunomodulator (e.g., vorinostat or resiquimod). In some embodiments of any of the aspects, the at least two immunomodulators are bexarotene and a second immunomodulator and are conjugated to the same a polymer (e.g., hyaluronic acid) molecule at a molar ratio of from 1:1 to 4:1 of bexarotene the second immunomodulator (e.g., vorinostat or resiquimod). In some embodiments of any of the aspects, the at least two immunomodulators are bexarotene and a second immunomodulator and are conjugated to the same a polymer (e.g., hyaluronic acid) molecule at a molar ratio of from about 1:1 to about 2:1 of bexarotene:the second immunomodulator (e.g., vorinostat or resiquimod). In some embodiments of any of the aspects, the at least two immunomodulators are bexarotene and a second immunomodulator and are conjugated to the same a polymer (e.g., hyaluronic acid) molecule at a molar ratio of from 1:1 to 2:1 of bexarotene:the second immunomodulator (e.g., vorinostat or resiquimod).

In some embodiments of any of the aspects, the at least one immunomodulator (collectively or individually) is present in the composition at a concentration of about 1.0 to about 50.0 mol %. In some embodiments of any of the aspects, the at least one immunomodulator (collectively or individually) is present in the composition at a concentration of 1.0 to 50.0 mol %. In some embodiments of any of the aspects, the at least one immunomodulatory (collectively or individually) is present in the composition at a concentration of about 2.0 to about 25.0 mol %. In some embodiments of any of the aspects, the at least one immunomodulator (collectively or individually) is present in the composition at a concentration of 2.0 to 25.0 mol %. In some embodiments of any of the aspects, the at least one immunomodulator (collectively or individually) is present in the composition at a concentration of about 4.0 to about 10.0 mol %. In some embodiments of any of the aspects, the at least one immunomodulator (collectively or individually) is present in the composition at a concentration of 4.0 to 10.0 mol %.

The compositions described herein can be provided as nanoparticles, microparticles, liposomes, and/or vesicles.

In some embodiments of any of the aspects, a composition and/or conjugate as disclosed herein, or a pharmaceutically acceptable salt or solvate thereof can be administered, e.g., topically, in a vesicle, in particular a liposome (see Langer, “New Methods of Drug Delivery,” Science 249: 1527-1533 (1990); Lopez-Berestein, “Treatment of Systemic Fungal Infections with Liposomal-Amphotericin B,” Liposomes in the Therapy of Infectious Disease and Cancer, pp. 317-327 (1989); and Treat et al, “Liposome encapsulated doxorubicin—preliminary results of phase I and phase II trials” Liposomes in the Therapy of Infectious Disease and Cancer, pp. 353-365 (1989). Compositions and/or conjugates as disclosed herein invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art. See, for example, Prescott, Ed., Methods in Cell Biology, Volume XIV, Academic Press, New York, N.Y. (1976), p. 33 et seq.

In some embodiments of any of the aspects, a composition and/or conjugate as disclosed herein, or a pharmaceutically acceptable salt or solvate thereof can be administered via a nanoparticle or microparticle, e.g., nanoparticles and microparticles formulated for topical drug delivery, as disclosed in Prow et at al, Nanoparticles and microparticles for skin drug delivery, Advanced Drug Delivery Reviews, 2011, 63(6), 470-491, which is incorporated herein in its entirety by reference.

The compositions described herein can modulate the phenotype of a macrophage, e.g., they can polarize macrophages and/or monocytes to an M1 phenotype. The presence of the composition in or on a cell can, by contacting the cell with the immunomodulator(s), direct or regulate the phenotype of the cell, e.g., increase the likelihood, duration, magnitude, or rate of development M1 (as opposed to M2) phenotypic characteristics. This can be referred to herein as “M1 polarization.” In some embodiments of any of the aspects, the macrophage is substantially driven to an M1 phenotype by contact with the composition described herein.

The compositions described herein can also display excellent delivery kinetics when they are administered in combination with certain cells, essentially using the cell as a delivery vector. Accordingly, in some aspects of any of the embodiments, provided herein is a cellular composition comprising a polymer (e.g., hyaluronic acid) conjugated to at least one immunomodulator, a) adhered to or located on the surface of a macrophage or monocyte, or b) present in a macrophage or monocyte. In some aspects of any of the embodiments, provided herein is a cellular composition comprising a polymer (e.g., hyaluronic acid) conjugated to at least two immunomodulators, a) adhered to or located on the surface of a macrophage or monocyte, or b) present in a macrophage or monocyte. In some aspects of any of the embodiments, provided herein is a cellular composition comprising a composition described herein (e.g., comprising a polymer (e.g., hyaluronic acid) and one or more immunomodulators in any arrangement described herein), a) adhered to or located on the surface of a macrophage or monocyte, or b) present in a macrophage or monocyte.

In one aspect of any of the embodiments, described herein is a method of increasing the activity of level of a M1 macrophage phenotype and/or decreasing the activity or level of a M2 macrophage phenotype, the method comprising contacting at least one macrophage with a composition described herein.

An M1 or M1-polarized macrophage, also referred to as “killer” macrophages, promotes inflammation and have anti-tumor activity. They secrete high levels of IL-12 and low levels of IL-10. M1 macrophages can be characterized by the expression of, e.g., CCL3, CCL5, CD80, CCR7, iNOS and INF-γ. In contrast, a M2 or M2-polarized macrophage, also referred to as a “repair” macrophage, contributes to wound healing and tissue repair. M2 macrophages can suppress the immune system and/or inflammation, e.g., by producing high levels of IL-10. An M2-polarized macrophage can be characterized by the expression of, e.g., CCL22, CD206, CD163, YM1, Fizz1, and arginase 1.

In some embodiments of any of the aspects, the compositions described herein can further comprise one or more M1 polarizing agents. As described herein, a “polarizing agent” is an agent, that when contacted with a macrophage and/or monocyte, alters the likelihood, persistence, magnitude, or rate of development of a particular macrophage phenotype (e.g., either M1 or M2 phenotype) as compared to the absence of the polarizing agent. A polarizing agent can be an M1-polarizing agent, e.g., it increases the likelihood, persistence, or rate of development of an M1 phenotype. Exemplary M1 and M2 phenotypes are described herein and are well known in the art. Further details can be found, e.g., in Mills et al. “M1/M2 macrophages” Frontiers Media SA (2015) and Kloc “Macrophages: Origin, Function, and Biointervention” Spring (2017); each of which are incorporated by reference herein in their entireties.

Polarizing agents for the M1 macrophage phenotypes are known in the art, and can include, by way of non-limiting example, the M1-polarizing Toll-like receptor (TLR) agonists (e.g., LPS, muramyl dipeptide, or lipoteichoic acid); the M1-polarizing cytokines IFN-γ (e.g., NCBI Gene ID: 3458); TNF (e.g., NCBI Gene ID: 7124); IL-12 (e.g., NCBI Gene ID: 3592 and 3593); GM-CSF (eg., NCBI Gene ID: 1438); IL-10 (e.g., NCBI Gene ID: 3553); IL-6 (e.g., NCBI Gene ID: 3569); CD11b (e.g., NCBI Gene ID: 3684) and IL-23 (e.g., NCBI Gene ID: 51561). TLR agonists are known in the art and can include, by way of non-limiting example LPS, dsRNA; flagella; bacterial lipoprotein; ssRNA; cpG DNA; bacterial peptidoglycans; profillin; rRNA; IMO-2055; picibanil; monophsophoryl lipid A (MPL); polyribocytidylic acid (polyL:C); CpG-28; MGN1703; glucopyranosyl lipid A; entolimod; and ODN2006. Further details on TLR agonists can be found, e.g., in Kaczanowska et al. 2013 J. Leukoc. Biol. 93:847-863; which is incorporated by reference herein in its entirety. TLR agonists are also available commercially, e.g., TLR1-9 Agonist Kit (Cat. No. tlrl-kitlhw; Invitrogen; San Diego, Calif.).

In some embodiments of any of the aspects, the composition described herein can be adhered to a cell by providing the composition in a cell-adherent polymeric particle, e.g., as described in International Patent Publication WO 2019/139892; which is incorporated by reference herein in its entirety. For example, a polymeric particle can comprise one or more cell adhesive mocules and one or more structural polymers (e.g., PVA, PLCL, PLGA, and/or PGS). Exemplary cell adhesive molecules can include polyelectrolytes, immunoglobulins, ligands for receptors on a cell surface, and/or monocyte-targeting and/or macrophage-targeting ligands; poly (glycidyl methacrylate) (PGMA); polycaprolactone (PCL); polydimethylsiloxane (PDMS); poly(hexamethyldisiloxane) (PHMDSO); superhydrophobic perfluoro-substituted PEDOT (PEDOT-F); superhydrophobic polystyrene (PS); plasma-treated poly (methyl methacrylate) (PMMA); plasma-treated poly-3-hydroxybutyrate (P3HB); phosphatidylethanolamine (PE); carboxymethyl chitin (CMCH4); RGD peptides, collagen, fibronectin, gelatin, and collagen. Further discussion of cell adhesive molecules can be found, e.g., at Lih et al. Progress in Polymer Science 44:28-61 (2015) and Chen et al. Materials Today (2017); which are incorporated by reference herein in their entireties. Exemplary structural polymers can include polylactide (PLA): polyglycolide (PGA); poly-(ε-caprolactone) (PCL); polyphosphazenes; polyorthoesters; polyanhydrides; poly(α-hydroxy esters); poly(ether esters); copolymers comprising lactide of glycolide and α-caprolactone or trimethylene carbonate; poly(polyol sebacate) elastomers; elastomers; poly(polyol citrate); polyesters; poly(glycolic acid); poly(lactic acid); poly(caprolactone); poly(lactic-co-glycolic acid); poly(butylene succinate); poly(trimethylene carbonate); poly(β-dioxanone); poly(butylene terephthalate); poly(ester amide)s; Hybrane™ S1200; DegraPol™; polyurethanes; polyanhydrides; poly[(caboxyphenoxy) propane-sebacic acid]; polyphsophoesters; poly[bis(hydroxyethyl) terephthalate-ethyl orthophosphorylate/terephthaloyl chloride]; poly(ortho esters); poly(alkyl cyanoacrylates); poly(butyl cyanoacrylate); polyethers; poly(ethylene glycol); poly(amino acids); tyrosine derived polycarbonate; microbial polyesters; poly(β-hydroxyalkanoate); poly(hydroxybutyrate); poly(hydroxybutyrate-co-hydroxyvalerate); collagen; albumin; gluten; chitosan; hyaluronate; cellulose; alginate; and starch. Suitable structural polymers are discussed in more detail at, e.g., Bat et al. Regen. Med. 9:385-398 (2014) and Marin et al. Int. J. Nanomedicine 8:3071-3091 (2013); which are incorporated by reference herein in their entireties. In some embodiments of any of the aspects, the structural polymer comprises poly(lactic-co-glycolic) acid (PLGA), polyvinyl alcohol (PVA), hyaluronic acid (HA), gelatin, collagen and/or poly(glycerol sebacate) (PGS).

As used herein, the term “polymer” refers to oligomers, co-oligomers, polymers and copolymers, e.g., random block, multiblock, star, grafted, gradient copolymers and combination thereof. The average molecular weight of the polymer, as determined by gel permeation chromatography, can range from 500 to about 500,000, e.g., from 20,000 to about 500,000. Without limitation, any polymeric material known in the art can be used in the invention. Accordingly, in some embodiments of any of the aspects, the polymer is selected from the group consisting of polysaccharides, polypeptides, polynucleotides, copolymers of fumaric/sebacic acid, poloxamers, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), gelatin, collagen, silk, alginate, cellulose, poly-nucleic acids, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), hyaluron, heparin, agarose, pullulan, and copolymers, terpolymers, and copolymers comprising any combinations thereof.

In some embodiments of any of the aspects, the polymer is a biocompatible polymer. As used herein, the term “biocompatible” means exhibition of essentially no cytotoxicity or immunogenicity while in contact with body fluids or tissues. The term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also non-inflammatory when employed in situ.

Biodegradable polymers are disclosed in the art. Examples of suitable biodegradable polymers include, but are not limited to, linear-chain polymers such as polypeptides, polynucleotides, polysaccharides, polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), fumaric acid, sebacic acid, and copolymers, terpolymers including one or more of the foregoing. Other biodegradable polymers include, for example, gelatin, collagen, silk, chitosan, alginate, cellulose, poly-nucleic acids, etc.

Suitable non-biodegradable biocompatible polymers include, by way of example, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), Poloxamers (e.g., Pluronic such as Poloxamers 407 and 188), hyaluronic acid, heparin, agarose, Pullulan, and copolymers including one or more of the foregoing, such as ethylene/vinyl alcohol copolymers (EVOH).

In some embodiments of any of the aspects, the biocompatible polymer is a copolymer of polylactic acid and polyglycolic acid, poly(glycerol sebacate) (PGS), poly(ethylenimine), Pluronic (Poloxamers 407, 188), hyaluronic acid, heparin, agarose, or Pullulan.

In some embodiments of any of the aspects, the polymer is a homopolymer, a copolymer or a block polymer.

In some embodiments of any of the aspects, the polymer comprises side chains selected from the group consisting of amide or ester groups. In some embodiments of any of the aspects, the polymer is biodegradable, biocompatible, and non-toxic.

The compositions described herein are also efficacious when located intracellularly in the macrophage and/or monocyte. Accordingly, in some embodiments the cellular composition comprises a macrophage and/or monocyte which comprises the composition, e.g., in the intraceullar space. The composition can be engulfed, phagocytosed, or otherwise internalized.

In some embodiments of any of the aspects, the cell is a monocyte cell. In some embodiments of any of the aspects the cell is a monocyte cell at the time the composition is adhered to the cell (e.g., the cell, either under the influence of the composition, or independently thereof, may differentiate to a macrophage after adherence) or the cell is contacted with the composition. In some embodiments of any of the aspects, the cell is a macrophage cell, e.g., an M0, M1, or M1-polarized, macrophage. In some embodiments of any of the aspects, the macrophage and/or monocyte is obtained from a subject. In some embodiments of any of the aspects, the macrophage and/or monocyte is autologous to a subject, e.g., to a subject administered the composition described herein.

The cellular compositions can comprise cells which are autologous to or heterologous to the subject to be treated. In some embodiments of any of the aspects, the method of treatment can comprise a first step of obtaining the cell from a donor and/or the subject and contacting the cell with the composition described herein ex vivo. The cell can be isolated, e.g., isolated from a blood sample obtained from the donor/subject prior to performing the contacting/adhering step, or the contacting/adhering can take place in a sample comprising multiple cell types, e.g., in a blood sample.

In one aspect of any of the embodiments, described herein is a method of stimulating an immune response in a subject in need thereof, the method comprising administering a composition as described herein to the subject. As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an a disease, an antigen, or healthy cells, e.g., in the case of autoimmunity). In some embodiments of the aspects described herein, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. Stimulation of an immune response refers to an induction or increase of the immune response. Suppression of an immune response refers to an elimination or decrease of the immune response.

An immune response can be the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+ T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The stimulation of a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments of any of the aspects, a subject in need of stimulation is a subject in need of treatment for cancer. In one aspect of any of the embodiments, described herein is a method for treating cancer in a subject in need thereof, the method comprising administering a composition as described herein to the subject.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

In some embodiments of any of the aspects, the cancer is melanoma. In some embodiments of any of the aspects, the cancer is metastatic.

In some embodiments of any of the aspects, the methods described herein relate to treating a subject having or diagnosed as having cancer with a composition described herein. Subjects having cancer can be identified by a physician using current methods of diagnosing cancer. Symptoms and/or complications of cancer, e.g., melanoma, which characterize these conditions and aid in diagnosis are well known in the art and include but are not limited to, changes in moles and the development of new pigmented or unusually growths or areas of skin. Tests that may aid in a diagnosis of, e.g. melanoma include, but are not limited to, biopsy and physical examination. A family history of cancer, or exposure to risk factors for cancer can also aid in determining if a subject is likely to have cancer or in making a diagnosis of cancer.

The compositions and methods described herein can be administered to a subject having or diagnosed as having cancer. In some embodiments of any of the aspects, the methods described herein comprise administering an effective amount of compositions described herein, e.g. a composition as described herein to a subject in order to alleviate a symptom of a cancer. As used herein, “alleviating a symptom” is ameliorating any condition or symptom associated with the disease, e.g., cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

In some embodiments of any of the aspects, the composition is administered by intravenous or intratumoral injection. In some embodiments of any of the aspects, the composition comprises resiquimod and bexarotene and is administered by intravenous or intratumoral injection.

In some embodiments of any of the aspects, the composition is administered topically. In some embodiments of any of the aspects, the composition comprises vorinostat and bexarotene and is administered topically.

As demonstrated herein, the provided compositions increase the efficacy of the immunomodulators, e.g. individually and/or by synergy. Accordingly, use of the compositions described herein permit the use of lower doses of immunomodulators to achieve therapeutic efficacy, e.g., as compared to administration of free drugs. In some embodiments of any of the aspects, the at least one immunomodulator is present at, or administered at, one half or less of the dose used when the immunomodulator is provided as a free drug. In some embodiments of any of the aspects, the at least one immunomodulator is present at, or administered at, one third or less of the dose used when the immunomodulator is provided as a free drug. In some embodiments of any of the aspects, the at least one immunomodulator is present at, or administered at, one quarter or less of the dose used when the immunomodulator is provided as a free drug.

The term “effective amount” as used herein refers to the amount of composition as described herein needed to alleviate at least one or more symptom of the disease or disorder, and relates to a sufficient amount of pharmacological composition to provide the desired effect. The term “therapeutically effective amount” therefore refers to an amount of composition as described herein that is sufficient to provide a particular effect (e.g., anti-tumor effect) when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient(s) which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for macrophage activity, among others. The dosage can be determined by a physician and adjusted, as necessary, to suit observed effects of the treatment.

In some embodiments of any of the aspects, the technology described herein relates to a pharmaceutical composition comprising a composition as described herein (e.g., at least one immunomodulator conjugated to a polymer, e.g., hyaluronic acid), and optionally a pharmaceutically acceptable carrier. In some embodiments of any of the aspects, the active ingredients of the pharmaceutical composition comprise a composition as described herein (e.g., at least one immunomodulator conjugated to a polymer, e.g., hyaluronic acid). In some embodiments of any of the aspects, the active ingredients of the pharmaceutical composition consist essentially of a composition as described herein (e.g., at least one immunomodulator conjugated to a polymer, e.g., hyaluronic acid). In some embodiments of any of the aspects, the active ingredients of the pharmaceutical composition consist of a composition as described herein (e.g., at least one immunomodulator conjugated to a polymer, e.g., hyaluronic acid). Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent.

In some embodiments of any of the aspects, the pharmaceutical composition comprising a composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of the compositions as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt as disclosed herein can also be incorporated into the parenteral dosage forms of the disclosure, including conventional and controlled-release parenteral dosage forms.

Pharmaceutical compositions described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif USA)), or a combination thereof to provide the desired release profile in varying proportions.

In some embodiments of any of the aspects, the composition described herein is administered as a monotherapy, e.g., another treatment for the cancer is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

In certain embodiments, an effective dose of a composition as described herein can be administered to a patient once. In certain embodiments, an effective dose of a composition as described herein can be administered to a patient repeatedly. For systemic administration, subjects can be administered a therapeutic amount of a composition as described herein such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. tumor growth or size by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to an active ingredient. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition as described herein can be administered over a period of time, such as over a 5 minute, 10 minute, 15 minute, 20 minute, or 25 minute period.

The dosage ranges for the administration of a composition as described herein, according to the methods described herein depend upon, for example, the form of the composition, its potency, and the extent to which symptoms, markers, or indicators of a condition described herein are desired to be reduced, for example the percentage reduction desired for tumor growth or size or the extent to which, for example, immune responses are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein (e.g. M1 polarization or immune activity) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor growth or size, or immune activity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. tumor inhibition or immune activity). It is well within the ability of one skilled in the art to monitor efficacy of administration and/or treatment by measuring any one of such parameters, or any combination of parameters. Efficacy can be assessed in animal models of a condition described herein, for example treatment of cancer in mice. When using an experimental animal model, efficacy of treatment is evidenced when a statistically significant change in a marker is observed, e.g. tumor size, tumor growth, or markers of immune activity.

In vitro and animal model assays are provided herein which allow the assessment of a given dose of a composition described herein. The efficacy of a given dosage combination can also be assessed in an animal model, e.g. a mouse model of cancer.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to tumor metastasis or remission. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

In some embodiments, the present technology may be defined in any of the following numbered paragraphs:
1. A composition comprising a polymer conjugated to at least one immunomodulator.
2. The composition of paragraph 1, wherein the polymer is hyaluronic acid.
3. The composition of any of the preceding paragraphs, wherein the immunomodulator is a retinoid, a HDAC inhibitor, or an imidazoquinoline.
4. The composition of any of the preceding paragraphs, wherein the immunomodulator is selected from the group consisting of: bexarotene, vorinostat, or resiquimod.
5. The composition of any of the preceding paragraphs, wherein the composition comprises at least two immunomodulators.
6. The composition of any of the preceding paragraphs, wherein the composition comprises at least a) a retinoid and an imidazoquinoline, b) a retinoid and a HDAC inhibitor, or c) a HDAC inhibitor and an imidazoquinoline.
7. The composition of any of the preceding paragraphs, wherein the composition comprises at least a) a retinoid and an imidazoquinoline or b) a retinoid and a HDAC inhibitor.
8. The composition of any of the preceding paragraphs, wherein the composition comprises resiquimod and bexarotene.
9. The composition of any of the preceding paragraphs, wherein the composition comprises vorinostat and bexarotene.
10. The composition of any of paragraphs 5-9, wherein the at least two immunomodulators are conjugated to the same polymer molecule such that each polymer molecule comprises at least one molecule of each of the at least two immunomodulators.
11. The composition of any of paragraphs 5-9, wherein the at least two immunomodulators are each conjugated to separate polymer molecules such that each polymer molecule comprises only one of the at least two immunomodulators.
12. The composition of any of paragraphs 5-9, wherein the at least two immunomodulators are present in equimolar amounts.
13. The composition of any of paragraphs 5-9, wherein the at least two immunomodulators are present in a ratio of from 1:2 to 2:1.
14. The composition of paragraph 13, wherein the at least two immunomodulators are present in a ratio of from 1:1 to 2:1 of bexarotene:a second immunomodulator.
15. The composition of any of the preceding paragraphs, wherein the at least one immunomodulator is conjugated directly to the polymer.
16. The composition of paragraph 15, wherein the conjugation is via an ester bond and/or via an amide bond.
17. The composition of any of the preceding paragraphs, wherein each polymer molecule is about 5 kDa to about 250 kDa in size.
18. The composition of any of the preceding paragraphs, wherein each polymer molecule is about 50 kDa in size.
19. The composition of any of the preceding paragraphs, wherein the at least one immunomodulator is present at a concentration of about 2.0 to about 25.0 mol %.
20. The composition of any of the preceding paragraphs, wherein the at least one immunomodulator is present at a concentration of about 4.0 to about 10.0 mol %.
21. The composition of any of the preceding paragraphs, wherein the polymer is hyaluronic acid and the composition does not comprise another polymer.
22. A cellular composition comprising a composition of any of paragraphs 1-21, a) adhered to or located on the surface of a macrophage or monocyte, or b) present in a macrophage or monocyte.
23. The cellular composition of paragraph 22, wherein the macrophage or monocyte is obtained from a subject.
24. The cellular composition of any of paragraphs 22-23, wherein the macrophage or monocyte is autologous to a subject.
25. A pharmaceutical composition comprising the composition or cellular composition of any of paragraphs 1-24 and optionally a pharmaceutically acceptable carrier.
26. A method of increasing the activity of level of a M1 macrophage phenotype and/or decreasing the activity or level of a M2 macrophage phenotype, the method comprising contacting at least one macrophage with a composition of any of paragraphs 1-25.
27. A method of stimulating an immune response in a subject in need thereof, the method comprising administering a composition of any of paragraphs 1-25 to the subject.
28. The method of any of paragraphs 26-27, wherein the subject is in need of treatment for cancer.
29. The method of paragraph 28, wherein the cancer is melanoma.
30. The method of any of paragraphs 28-29, wherein the cancer is metastatic.
31. The method of any of paragraphs 26-30, wherein the composition is administered by intravenous or intratumoral injection.
32. The method of paragraph 31, wherein the composition comprises resiquimod and bexarotene and is administered by intravenous or intratumoral injection.
33. The method of any of paragraphs 26-30, wherein the composition is administered topically.
34. The method of paragraph 33, wherein the composition comprises vorinostat and bexarotene and is administered topically.
35. The composition of any of paragraphs 1-25 for use in a method of stimulating an immune response in a subject in need thereof, the method comprising administering the composition to the subject.
36. The composition of any of paragraphs 1-25 for use in a method of treating cancer in a subject in need thereof, the method comprising administering the composition to the subject.
37. The composition of paragraph 36, wherein the cancer is melanoma and/or metastatic.
38. The composition of any of paragraphs 35-37, wherein the composition is administered by intravenous or intratumoral injection.
39. The composition of paragraph 38, wherein the composition comprises resiquimod and bexarotene and is administered by intravenous or intratumoral injection.
40. The composition of any of paragraphs 35-37, wherein the composition is administered topically.
41. The composition of paragraph 40, wherein the composition comprises vorinostat and bexarotene and is administered topically.
42. The method or composition of any of the preceding paragraphs, wherein the at least one immunomodulator is present at, or administered at, one half or less of the dose used when the immunomodulator is provided as a free drug.

In some embodiments, the present technology may be defined in any of the following numbered paragraphs:
1. A composition comprising a polymer conjugated to at least one immunomodulator.
2. The composition of paragraph 1, wherein the polymer is hyaluronic acid.
3. The composition of any of the preceding paragraphs, wherein the immunomodulator is a retinoid, a HDAC inhibitor, or an imidazoquinoline.
4. The composition of any of the preceding paragraphs, wherein the immunomodulator is selected from the group consisting of: bexarotene, vorinostat, or resiquimod.
5. The composition of any of the preceding paragraphs, wherein the composition comprises at least two immunomodulators.
6. The composition of any of the preceding paragraphs, wherein the composition comprises at least a) a retinoid and an imidazoquinoline, b) a retinoid and a HDAC inhibitor, or c) a HDAC inhibitor and an imidazoquinoline.
7. The composition of any of the preceding paragraphs, wherein the composition comprises at least a) a retinoid and an imidazoquinoline or b) a retinoid and a HDAC inhibitor.
8. The composition of any of the preceding paragraphs, wherein the composition comprises resiquimod and bexarotene.
9. The composition of any of the preceding paragraphs, wherein the composition comprises vorinostat and bexarotene.
10. The composition of any of paragraphs 5-9, wherein the at least two immunomodulators are conjugated to the same polymer molecule such that each polymer molecule comprises at least one molecule of each of the at least two immunomodulators.
11. The composition of any of paragraphs 5-9, wherein the at least two immunomodulators are each conjugated to separate polymer molecules such that each polymer molecule comprises only one of the at least two immunomodulators.
12. The composition of any of paragraphs 5-9, wherein the at least two immunomodulators are present in equimolar amounts.
13. The composition of any of paragraphs 5-9, wherein the at least two immunomodulators are present in a ratio of from 1:2 to 2:1.
14. The composition of paragraph 13, wherein the at least two immunomodulators are present in a ratio of from 1:1 to 2:1 of bexarotene:a second immunomodulator.
15. The composition of any of the preceding paragraphs, wherein the at least one immunomodulator is conjugated directly to the polymer.
16. The composition of paragraph 15, wherein the conjugation is via an ester bond and/or via an amide bond.
17. The composition of any of the preceding paragraphs, wherein each polymer molecule is about 5 kDa to about 250 kDa in size.
18. The composition of any of the preceding paragraphs, wherein each polymer molecule is about 50 kDa in size.
19. The composition of any of the preceding paragraphs, wherein the at least one immunomodulator is present at a concentration of about 2.0 to about 25.0 mol %.
20. The composition of any of the preceding paragraphs, wherein the at least one immunomodulator is present at a concentration of about 4.0 to about 10.0 mol %.
21. The composition of any of the preceding paragraphs, wherein the polymer is a hydrophilic polymer.
22. The composition of any of the preceding paragraphs, wherein the polymer comprises one or more of hyaluronic acid; polygutamic acid; chitosan; dextran; and a polyvinyl alcohol.
23. The composition of any of the preceding paragraphs, wherein the polymer comprises hyaluronic acid.
24. The composition of any of the preceding paragraphs, wherein the polymer is hyaluronic acid and the composition does not comprise another polymer.
25. A cellular composition comprising a composition of any of paragraphs 1-24, a) adhered to or located on the surface of a macrophage or monocyte, or b) present in a macrophage or monocyte.
26. The cellular composition of paragraph 25, wherein the macrophage or monocyte is obtained from a subject.
27. The cellular composition of any of paragraphs 25-26, wherein the macrophage or monocyte is autologous to a subject.
28. A pharmaceutical composition comprising the composition or cellular composition of any of paragraphs 1-27 and optionally a pharmaceutically acceptable carrier.
29. A method of increasing the activity of level of a M1 macrophage phenotype and/or decreasing the activity or level of a M2 macrophage phenotype, the method comprising contacting at least one macrophage with a composition of any of paragraphs 1-28.
30. A method of stimulating an immune response in a subject in need thereof, the method comprising administering a composition of any of paragraphs 1-28 to the subject.
31. The method of any of paragraphs 29-30, wherein the subject is in need of treatment for cancer.
32. The method of paragraph 31, wherein the cancer is melanoma.
33. The method of any of paragraphs 31-32, wherein the cancer is metastatic.
34. The method of any of paragraphs 30-33, wherein the composition is administered by intravenous or intratumoral injection.
35. The method of paragraph 34, wherein the composition comprises resiquimod and bexarotene and is administered by intravenous or intratumoral injection.
36. The method of any of paragraphs 30-33, wherein the composition is administered topically.
37. The method of paragraph 36, wherein the composition comprises vorinostat and bexarotene and is administered topically.
38. The composition of any of paragraphs 1-28 for use in a method of stimulating an immune response in a subject in need thereof, the method comprising administering the composition to the subject.
39. The composition of any of paragraphs 1-28 for use in a method of treating cancer in a subject in need thereof, the method comprising administering the composition to the subject.
40. The composition of paragraph 39, wherein the cancer is melanoma and/or metastatic.
41. The composition of any of paragraphs 38-40, wherein the composition is administered by intravenous or intratumoral injection.
42. The composition of paragraph 41, wherein the composition comprises resiquimod and bexarotene and is administered by intravenous or intratumoral injection.
43. The composition of any of paragraphs 38-40, wherein the composition is administered topically.
44. The composition of paragraph 43, wherein the composition comprises vorinostat and bexarotene and is administered topically.
45. The method or composition of any of the preceding paragraphs, wherein the at least one immunomodulator is present at, or administered at, one half or less of the dose used when the immunomodulator is provided as a free drug.

### EXAMPLES

### Example 1: Combination Conjugates of TLR7/8-Agonist and Third Generation Retinoid Promoted the Polarization of Tumor Associated Macrophages and Enhanced Antitumor Efficacy Against Melanoma

Introduction: Melanoma is one of the most aggressive types of skin cancer, causing a large majority of skin-cancer related deaths worldwide. It is treated by radiation, chemotherapy and surgery. However, treatment often end in recurrence. Hence, immunotherapy aiming at modulating the tumor microenviroment is gaining more interest than focusing on directly killing tumor cells. Tumor-associated macrophages (TAMs), are one of the most abundant tumor-infiltrating leukocytes in various tumors. Macrophages exist in a wide range of phenotypes with the anti-inflammatory (M2) phenotype on one side and pro-inflammatory classical (M1) phenotype on the other side. TAMs tend to polarize to an activated M2 rather than the activated M1 phenotype, thereby assisting tumor development by inducing immune suppression, and angiogenesis. Repolarization of TAMs from M2 toward M1 phenotype would help in enhancing the antitumor efficacy against cancer. Current immunotherapies focus on the use of a single immunomdulatory agent beside a check point inhibitor. This approach despite being effective still faces a lot of limitations due to toxicity related issues.

Resiquimod is a powerful immune response modifier that stimulates the body's own immune system to attack cancer cells by activating toll-like receptors 7/8. It promotes the polarization of tumour-associated macrophages toward an M1 phenotype. Despite its reported effectiveness in suppressing the growth of several types of cancer, currently it is only available in the market as topical gel. However topical application is not effective against deep cutaneous tumors as melanoma. Bexarotene (BEX) is a third generation retinoid that has the ability to diminish the myeloid-derived suppressor cells and to down regulate the activation of M2 macrophages. However, its poor solubility and poor bioavailability limits its clinical use. Described herein is the use hyaluronic acid (HA) conjugates for the codelivery of the two selected immunomodulatory agents.

Methods: The inventors optimized a synthetic procedure to conjugate a retinoid (BEX) and a TLR7/8-agonist (RES) to HA via ester and amide bonds respectively. The conjugates were characterized to ensure identity, and total drug loading. The effect of the conjugates on bone marrow derived macrophages was evaluated with respect to their viability, activity and phagocytic ability. The cytotoxic effect of the conjugates was also tested on B16F10 melanoma cell line. The therapeutic efficacy of the conjugates was evaluated in subcutaneous mouse melanoma model.

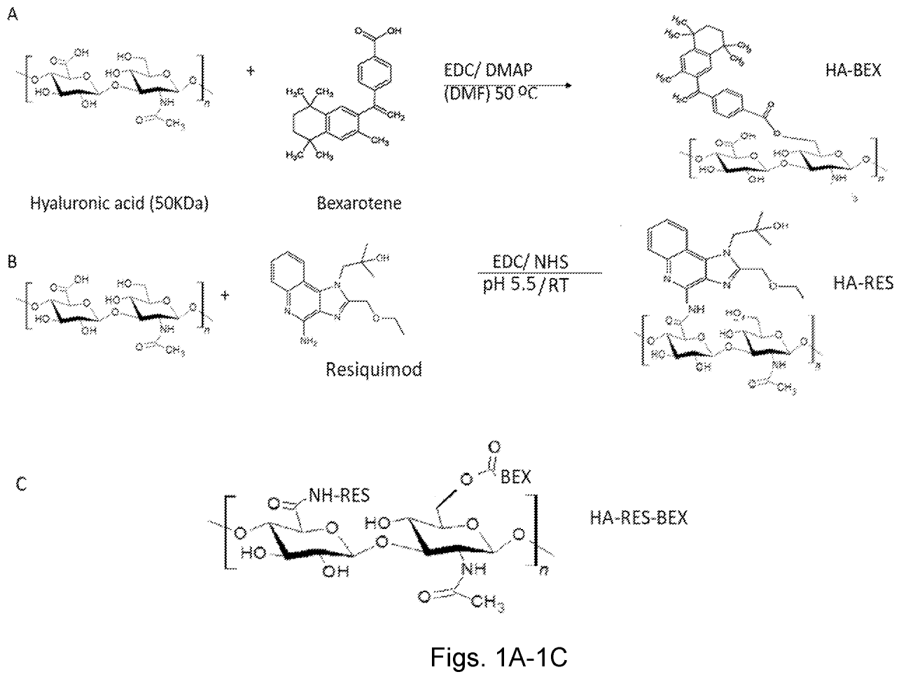

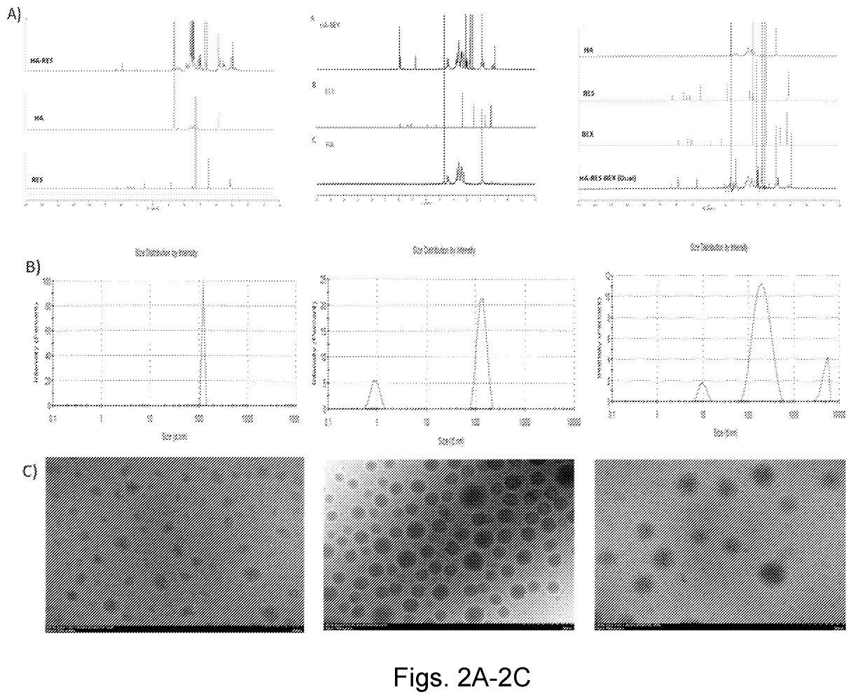

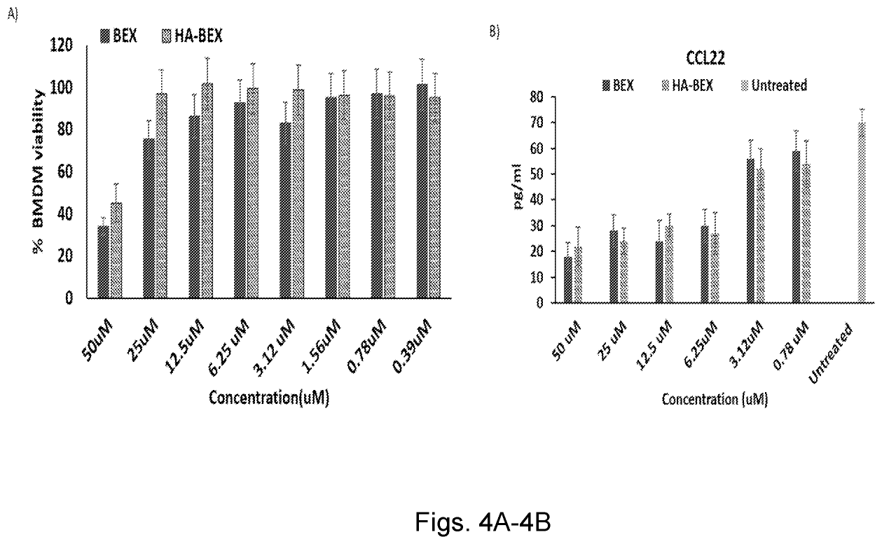

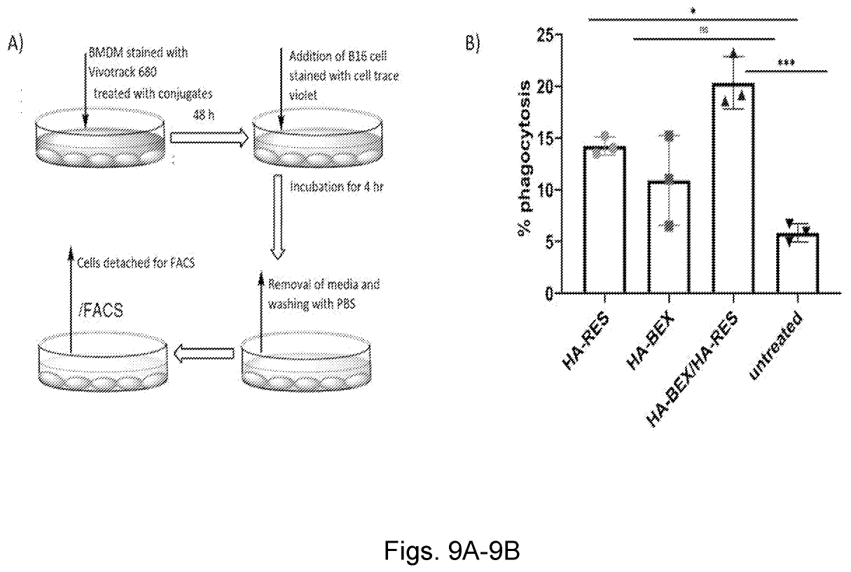

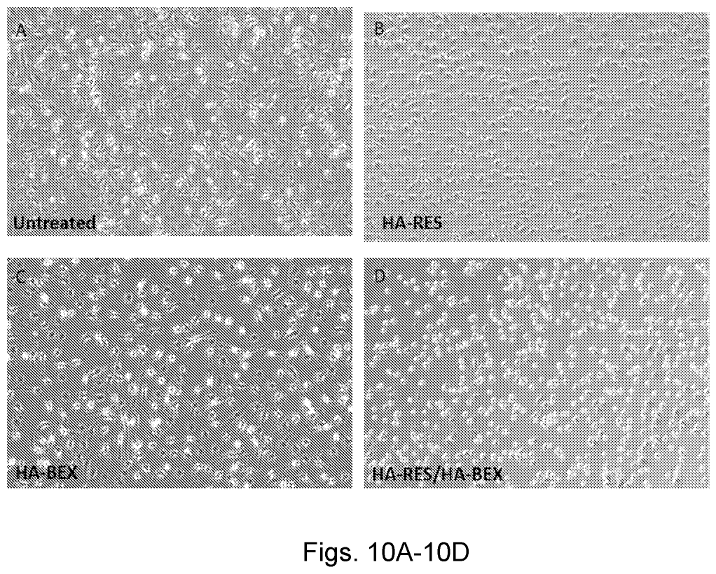

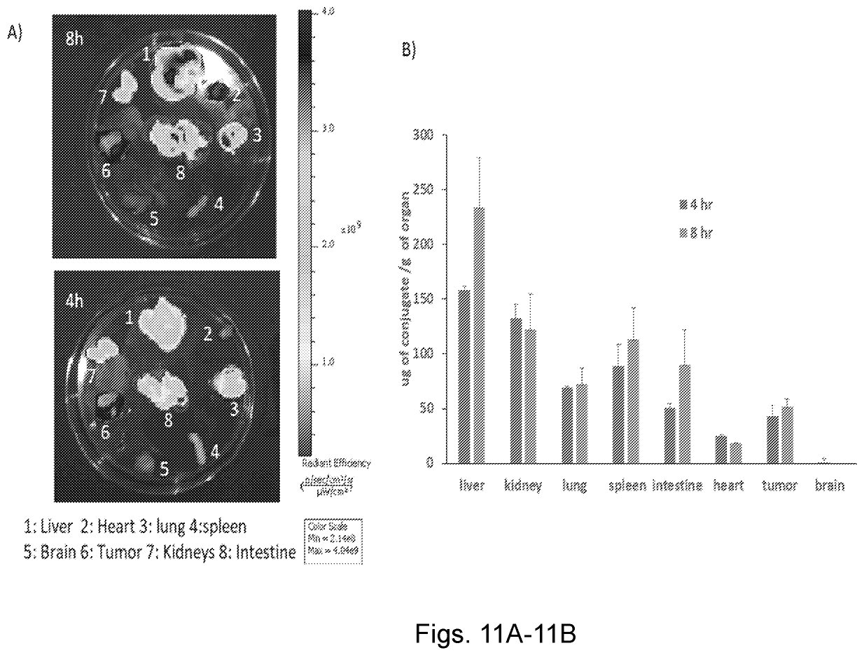

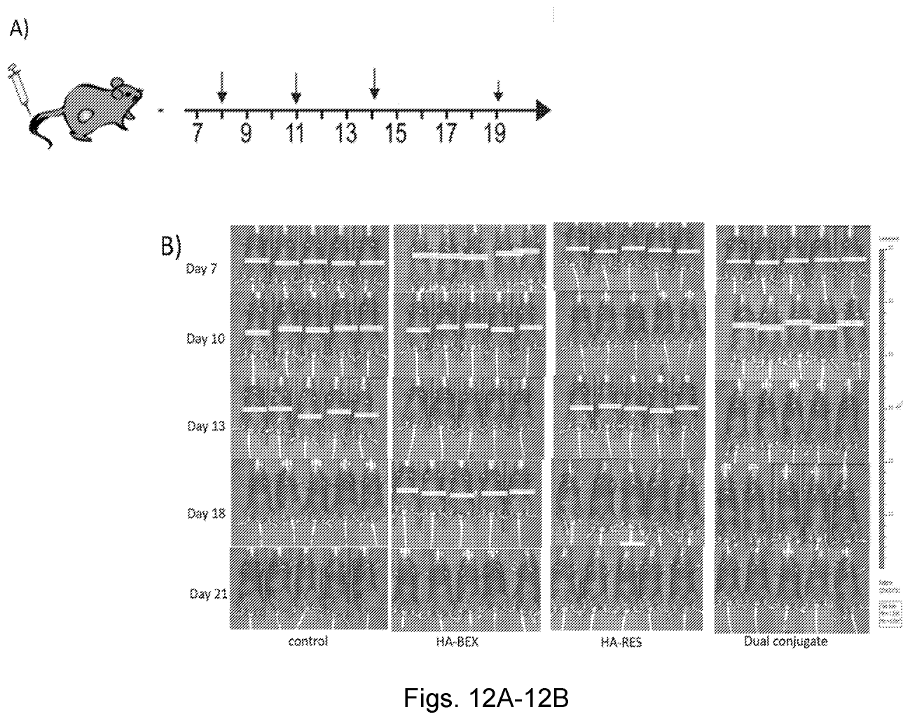

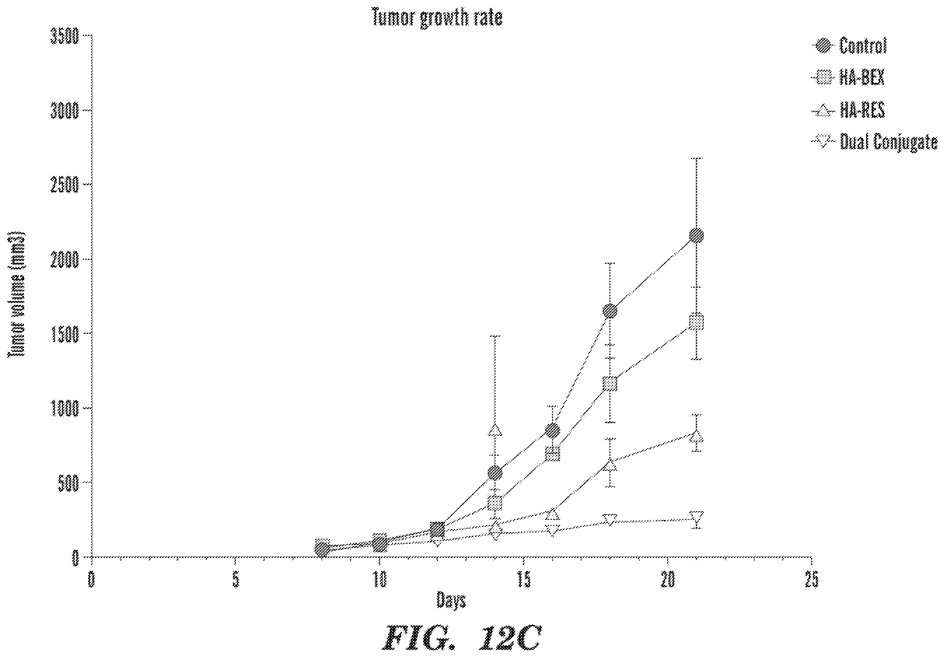

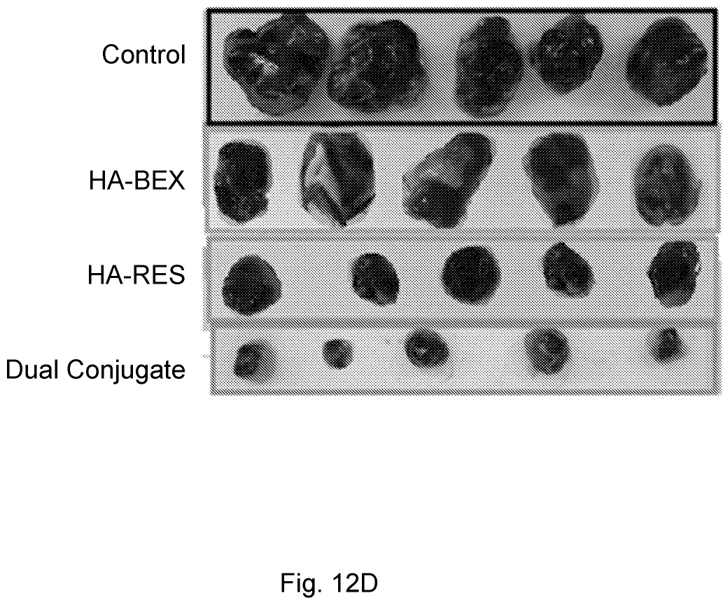

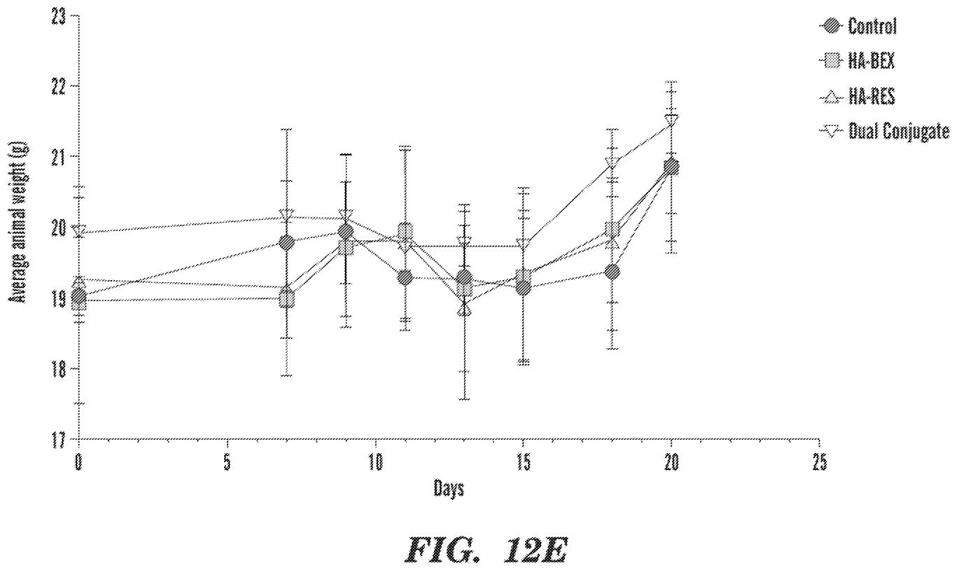

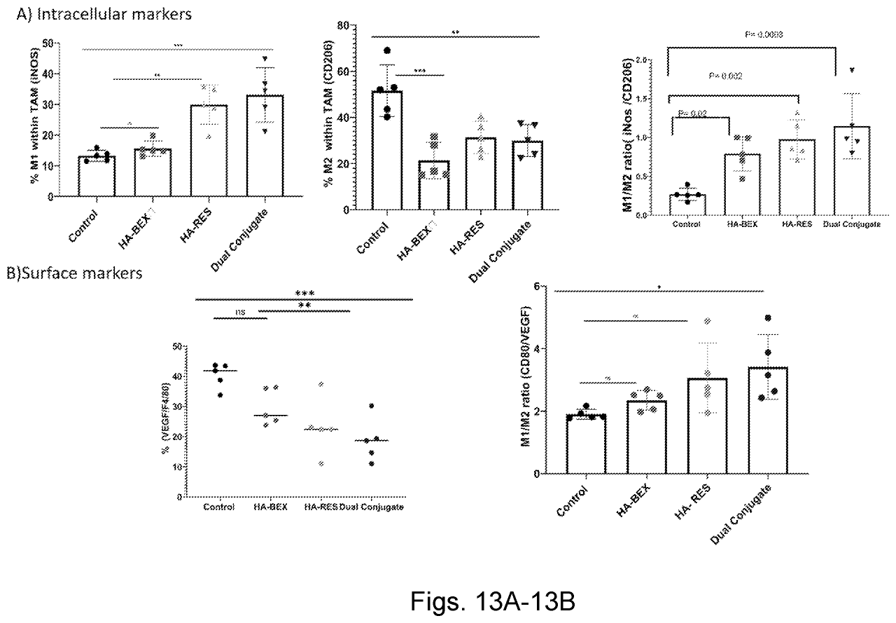

Results: The inventors designed and synthesized HA-conjugates that act as dual macrophage polarizer reserving the immunomodulatory effects of the parent drug molecules and showing enhanced inhibitory effect against melanoma cell line compared to the free drugs and the single HA-conjugates. Invivo studies confirmed the superiority of the dual conjugate to the single HA-drug conjugates in reducing the tumor size. Immunoprofiling of the excised tumors demonstrated a significant increase in the M1/M2 ratio in the group treated with the dual conjugate. (FIGS. 1A-13B).

Conclusion: The inventors have developed an injectable form of resiquimod and bexarotene based on HA-drug conjugates as an effective combination therapy for melanoma that promotes macrophage polarization towards an antitumorgenic phenotype and showed significant slowing in the tumor growth rate. This combination is effective against other forms of cancer as well particularly metastatic ones.

Biological Activity of HA-BEX and HA-RES

Immune cells system as macrophages and dendritic cells are responsible for the initiation of immune responses, and are the prime target for immunostimulatory drugs. To verify that the HA-RES conjugate is not toxic for immune cells, BMDM were treated with increasing concentrations of RES or HA-RES for 48 h. Both the free drug and its HA-conjugate did not have any negative effect on the BMDM but they caused marked increase in cell viability at all the concentrations tested. This effect is similar reports where RES was found to enhance the viability of bone marrow derived dendritic cells.

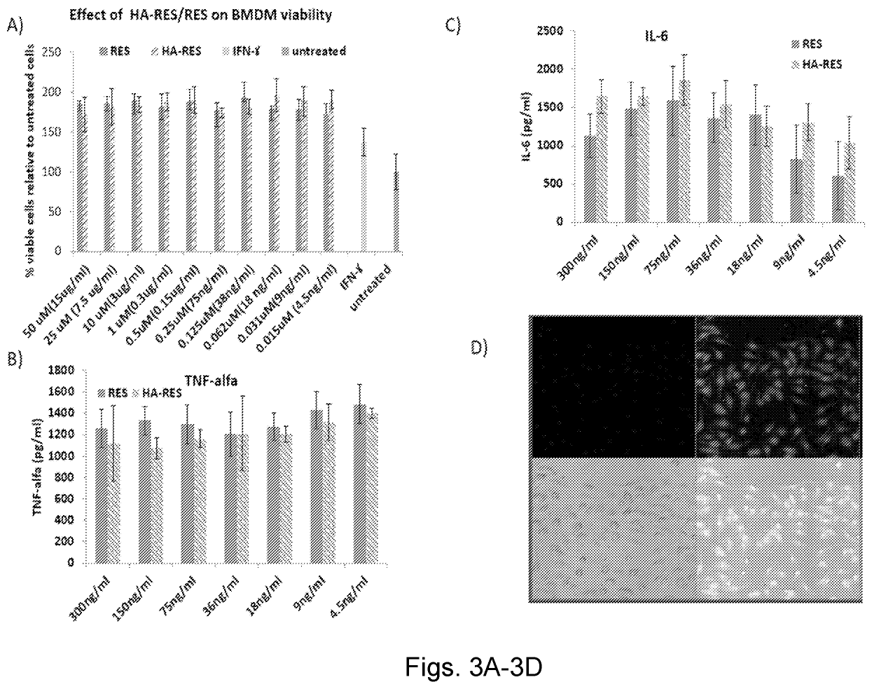

After achieving successful conjugation of RES to HA it was tested whether the conjugate still retained the immunostimulatory effects of the parent molecule. The inventors assessed the pro-inflammatory cytokines in the supernatants of BMDM after 48 h exposure to RES and HA-RES. The secretion of proinflammatory cytokines such as, TNF-α, IL-6 serve as quantitative indicators for TLR7 activation of BMDM. In these experiments, HA-RES clearly induced the secretion of the cytokines TNFα and IL-6. The induction of proinflammatory cytokines was similar for HA-RES and the equivalent amount of free RES, indicating that conjugation of RES to HA did not affect its immunological activity. Moreover, the levels of TNF-α and IL-6 detected where much higher than those produced by cells treated with IFN-γ (20 ng/ml). Also, since RES is a synthetic ligand of the endosomal receptor TLR7, delivery to the endosome of phagocytic cells is essential for its immunological activity. The inventors incubated fluorescently labelled HA-RES with BMDM for 4 h and observed endosomal localization of the conjugate (FIG. 3D).

The inventors also tested the effect of BEX and its HA conjugate on the viability of BMDM by exposing them to increasing concentration ranging from (0.39-50 μM). It was found that both the free BEX and its HA conjugate didn't alter the viability of the BMDM at all the concentrations tested up to 25 uM after which further increase the concentration to 50 uM caused significant decrease in the cells viability. The conjugate retain the ability of BEX to reduce the production of CCL22 from macrophages.

Interestingly, the co-administration of HA-RES with HA-BEX to BMDM did not have show any negative effect on the cell viability at all the concentrations tested. Even HA-BEX cone (50 uM) which showed reduction in BMDM viability did not show any inhibition of BMDM when combined with HA-RES. i.e the combination of HA-RES with HA-BEX even at the least conc of RES 0.03 μM enhanced MAC viability.

The inventors also measured the level of cytokines in the supernatant of BMDM after 48 h treatment with HA-RES, HA-BEX and their combinations. Interestingly, there was increase in the production of IL-6 and TNF-α while there was a significant reduction the production of CCL2 which is secreted by M2 macrophages. These results indicated that the combination of HA-Res and HA-BEX might act as a promising couple together by upregulating proinflammtory cytokines as IL-6 and TNF-α while inhibiting the production of CCl22 a cytokine whose down regulation plays a crucial role in suppressing melanoma growth.

BMDM Polarization

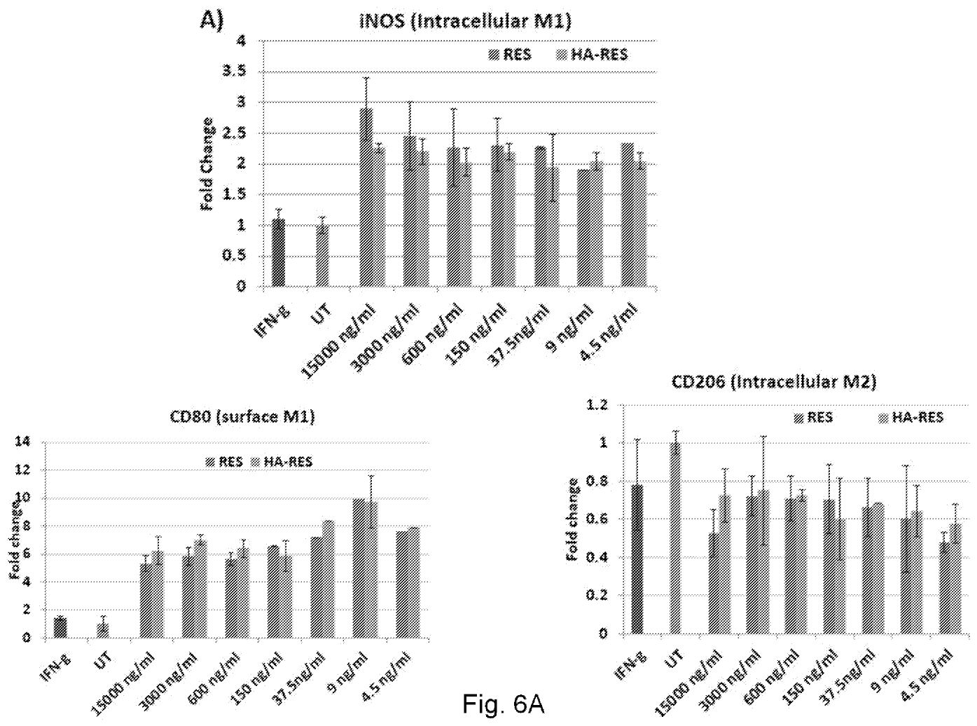

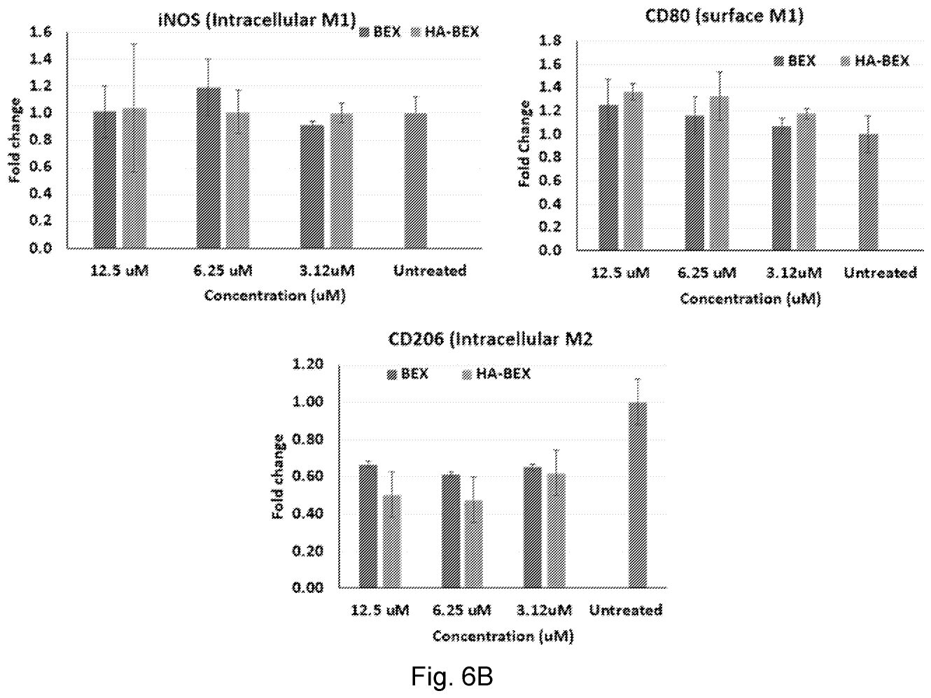

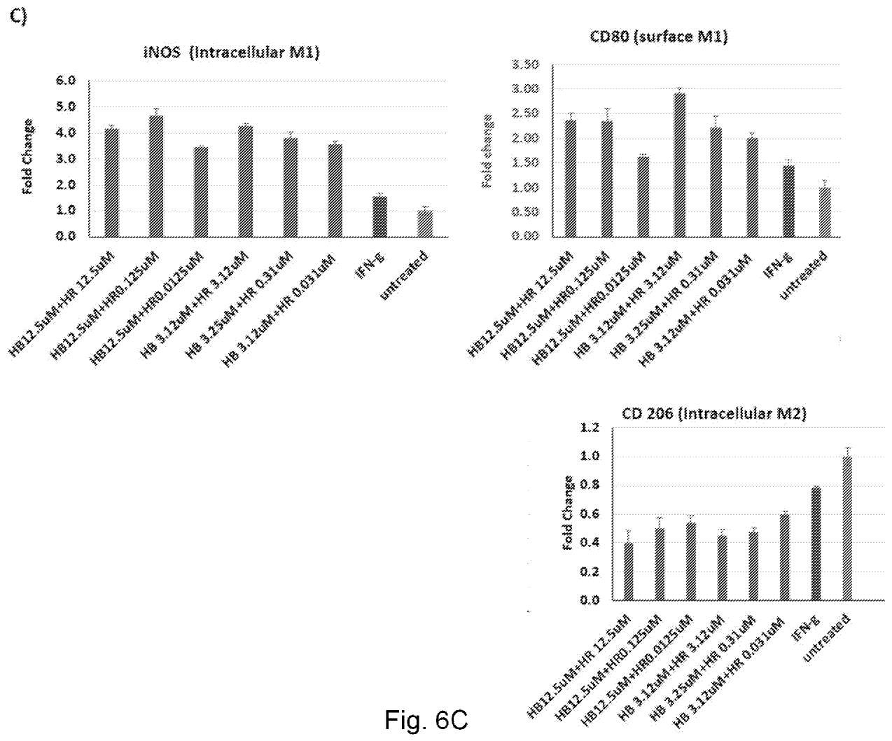

To further elucidate the effect of HA-RES and HA-BEX and their combination on macrophage phenotyping the inventors studied their effect on macrophage polarization. BMDMs were treated with different concentrations of RES and HA-RES for 48 hr. (FIG. 6A)

The expression of several markers associated with M1 and M2 phenotypes was evaluated. There was a high significant increase (p<0.0001) in the expression of CD80 and iNOS (M1 markers) in the cells treated with either free RES or HA-RES compared to the untreated cells.

Also a significant reduction (p<0.001) in CD206 was observed for cells treated with Res or HA-RES at all the investigated concentrations compared to the untreated cells. The inventors compared the level of marker expression to that induced by IFN-γ (a known M1 polarizer). Interestingly the fold change in the markers expression was higher than that induced by IFN-γ. This further confirmed that conjugating RES to HA did not affect its polarizing potential to BMDM. The effect was non concentration dependent.

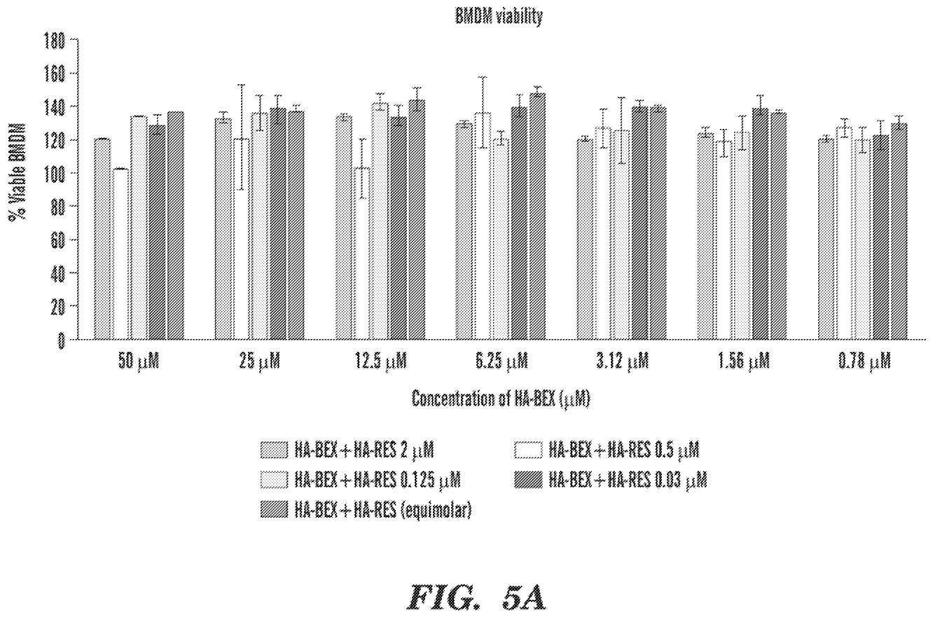

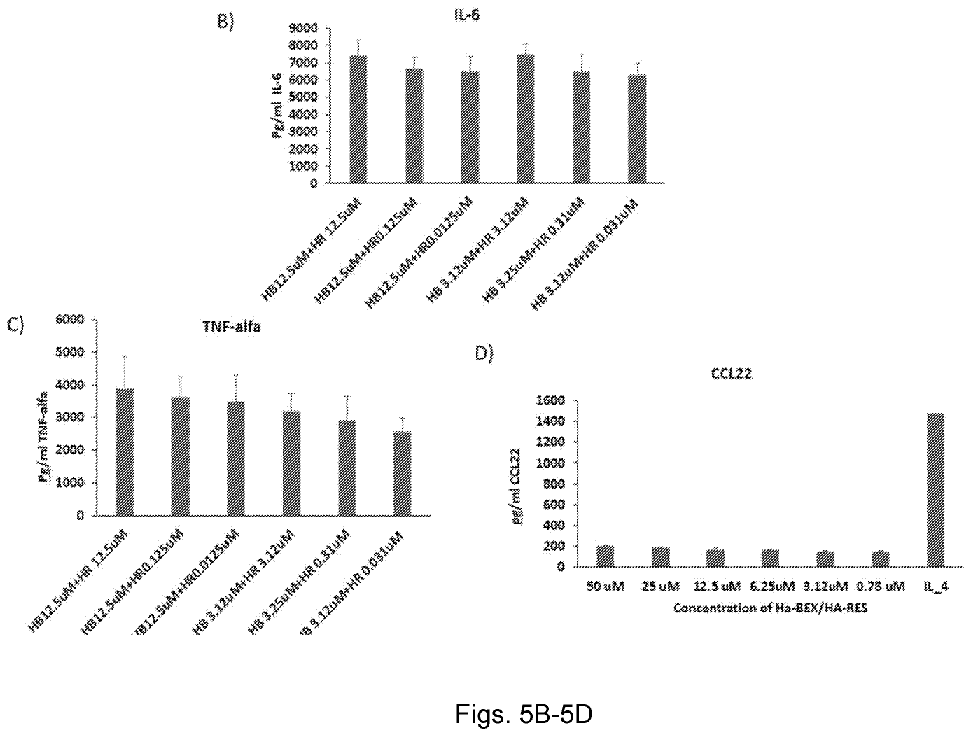

The inventors then studied the effect of BEX and HA-BEX on BMDMs polarization (FIG. 5B). Treatment with BMDMs with different concentrations of BEX or HA-BEX caused significant reduction in the expression of CD206 (M2 Marker). Treatment of BMDMs with a combination of HA-BEX and HA-RES at different ratios (1:1, 1:100, 1:1000) showed a very high significant increase in the expression of iNOS (M1 marker) at all the tested concentrations and ratios and a significant reduction in the expression of CD206 (M2 marker). The fold change in the marker expression was higher than that that observed for either HA-RES of HA-BEX alone. (FIG. 6C).

Cytotoxicity Study

5×10³ B16F10-Luc cells were seeded in 96 well plates and allowed to adhere overnight. For establishment for a coculture, B16F10 cells were cocultured with BMDM at a ratio of 1:10 (5×10³ B16F10: 5×10⁴ BMDM per well). The next day, the media was aspirated and replaced with increasing concentration of the free drugs, combination and the HA-conjugates with equivalent free drug concentrations and cells were incubated with the drug solutions for 48 h after which the treatment was aspirated and replaced with 0.5% Xenolight-D-luciferin potassium salt solution in complete DMEM. Finally, cellular viability was assessed by reading the luminescence of the solution from each well after 5 min using BioTek plate reader. Untreated cells were used as controls.

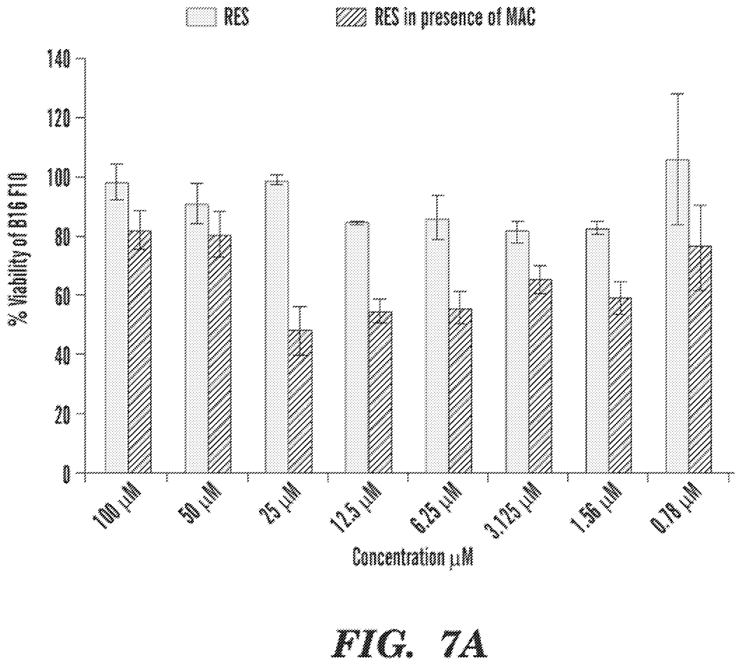

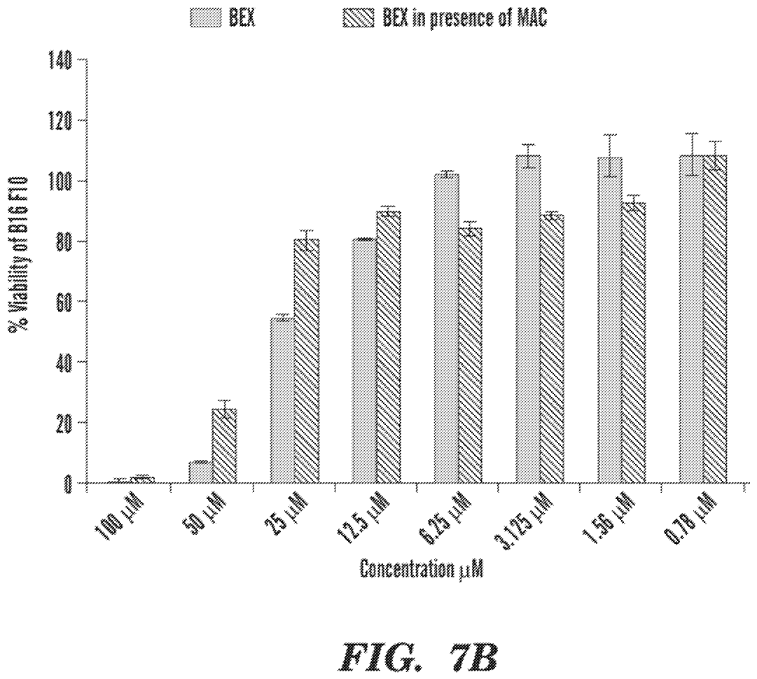

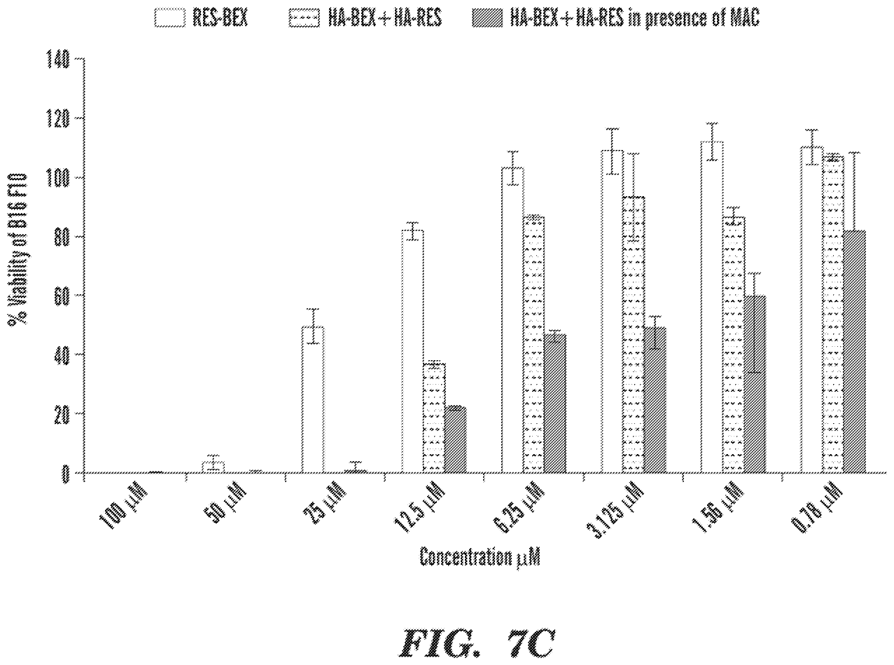

Resiquimod has a negligible cytotoxic effect on melanoma cells. Resiquimod effect on B16F10-Luc cells was significantly enhanced in the presence of macrophages (MAC). (FIG. 7A)

Bexarotene has an IC50 of 25 μM on B16 F10 (FIG. 7B). The Cytotoxic effect of BEX was not improved in the presence of MAC (same IC50).

The combination of free BEX and free RES did not result in any enhancement of cytotoxicity (same effect as BEX alone) (FIG. 7C). Interestingly, the combined conjugates were more potent in inhibiting the growth of the cancer cells. The IC50 for Bex on B16F10 cells is 25 uM. Adding free RES at equimolar concentration to BEX did not show any additional effect. However, combining both conjugates at the same conc results in 100% killing of the melanoma cells. The combination of the HA-BEX/HA-RES conjugates caused a 2.2 fold reduction in IC50 of BEX. (11.22 uM).

At a conc of 12.5 uM the free drug combination caused 13.6% inhibition of cell viability while the combined conjugates caused 62.7% inhibition in cell viability. A significant reduction of B16F10 viability was observed at all the tested concentrations of the combined conjugates in the coculture with BMDM (FIG. 7C).

Cellular Uptake

To understand the mechanism behind the enhanced cytotoxic effect of the combined conjugates against melanoma cells was compared to the free drugs, e.g., the quantitative uptake of the free drugs and the conjugates by BMDM. BMDM were seeded in sterile 6 well plates at a density of 1×10⁶ cell/well. 50 μM of each of free RES, free BEX,equivalent amount of HA-RES,HA-BEX. To study the effect of HA mediated uptake, cells were treated with free HA in an amount equal to that used in the conjugates for a period of 2 h followed by the addition of the conjugates. After 4 hr the supernatants were aspirated, the cells were washed twice with 2 ml HBSS then were treated with 2 ml of 0.5% solution of Triton X-100 for 3 h to lyse the cells. The lysate was centrifuged and the supernatant was analyzed for fluorescence on a on a Spectramax i3 plate reader for the quantification of RES.

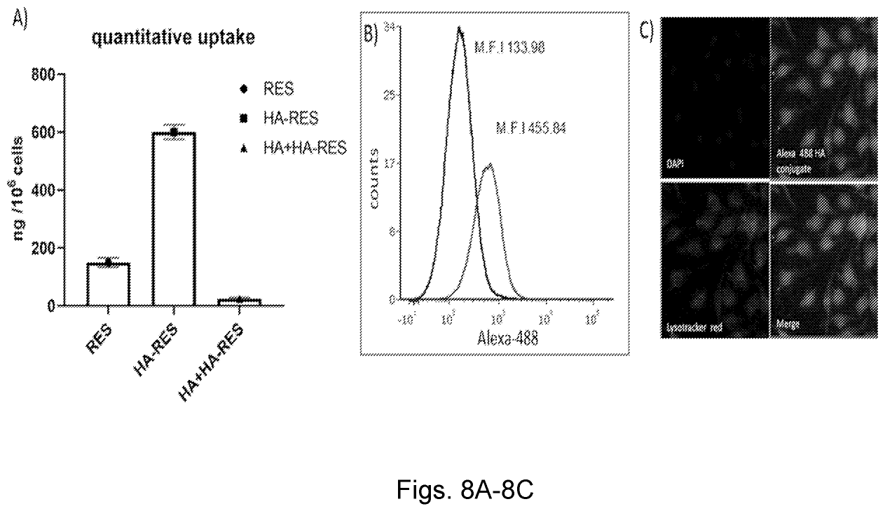

The uptake of HA-Res was 4 fold higher than free RES. However, upon pretreatment with HA, the uptake of HA-RES diminished (24 fold decrease). This indicates that the uptake is mediated through HA receptors (CD44) on the BMDM. Thus HA conjugate can permit the use of 4 fold lower dose of RES achieving the same amount of uptake by macrophages. (N.B. couldn't quantify HA-BEX due to interference from the blank in UV measurement) (FIG. 8A).

Phagocytosis Study

There are reports that the polarization of macrophages toward M1 phenotype enhances their phagocytic ability. Accordingly, the inventors performed phagocytosis study to understand the mechanism underlying the enhanced cytotoxic effect of the combined conjugates compared to the single conjugates in the coculture with BMDM.

Biodistribution Study

B16F10-Luc Melanoma cells (1×10⁶) were implanted subcutaneously in the fat pad of 6-8 weeks old C57BL/6 mice. 14 days after inoculation the mice were injected with 100 ul of 20 mg/ml HA-BEX-RES-Alexa 647 conjugate. The animals were euthanized after 4 and 8 h. Mice were euthanized at 4 and 8 hr post injection. Surgically resected organs were thoroughly washed with PBS, and imaged using IVIS filter set (excitation 640 nm and emission 680 nm). All the images were taken using a Perkin Elmer IVIS Spectrum CT In Vivo Imaging system. The organs were then weighed, homogenized in PBS using a high shear homogenizer and homogenates were crashed with ethanol, centrifuged and the supernatants were analyzed for florescence intensity using a calibration curve of the fluorescent HA conjugate in the organ homogenate.

In Vivo Antitumor Effect

B16F10-Luc (1×10⁶) were implanted subcutaneously in the flanks of 6-7 weeks old C57BL/6 mice (weighing 20 g, Charles River Laboratories). At day 8 after tumor inoculation the treatment was started. Mice were divided into four groups (n=5), receiving HA-RES, HA-BEX, dual HA conjugate and a control group (Saline). The mice were given a dose equivalent to 5 mg/kg body weight on days 8, 11, 14 and 19. The mice were imaged using in vivo imaging (PerkinElmer IVIS Spectrum, MA, USA) on days 7, 10, 13, 18 and 21. The tumor volumes and body weights were monitored every other day.

### Example 2:Hyaluronic Acid Conjugates of Vorinostat and Bexarotene for Treatment of Cutaneous Malignancies

Skin cancer is the most common class of malignancies in humans with increasing incidence worldwide. Although surgery remains the most common approach to treating skin cancer, relapses after surgery necessitate other treatment options. There is an increasing clinical demand for topical treatments for skin cancer that are capable of improving the drug's cutaneous localization while limiting its systemic absorption. The inventors successfully synthesized hyaluronic acid (HA) conjugates by attaching a third-generation retinoid (bexarotene) and a histone deacetylase inhibitor (vorinostat) to HA via ester bonds. The conjugates were characterized to ensure identity, purity and quantify the total drug loading. HA-BEX and HA-VOR exhibited a synergistic anticancer effect against human squamous cell carcinoma (A431) and human cutaneous T cell lymphoma cell lines (HUT 78) at specific ratios with minimal adverse effect on primary human keratinocytes and fibroblasts. Confocal microscopy demonstrated localization of the conjugates inside the cancer cells and ex vivo skin permeation studies on human skin explants confirmed their ability to penetrate the stratum corneum and reach the deep dermis. Biodistribution studies in the skin revealed the presence of both drugs in the epidermis and dermis in amounts sufficient to exert inhibitory effect on cancer cells. HA-bexarotene-vorinostat conjugates offer an excellent therapeutic option to treat skin cancers.

Introduction

The skin is the largest organ of the human body, acting as a protective barrier against the external environment. Skin cancer is the most common class of malignancy in humans accounting for over a million cases each year.^{[1]} Skin cancer can be divided into melanoma and non-melanoma skin cancers. The latter consists of basal cell carcinoma (BCC) and squamous cell carcinoma (SCC), both of which are very common. Additional forms of skin cancers include cutaneous lymphomas, Merkel cell carcinoma, and Kaposi sarcoma (KS). Although surgery remains the most common approach to treat skin cancer, relapses after surgery necessitate the use of other treatment options for the management of cutaneous neoplasms.

The systemic use of hormonal, immunosuppressive or cytotoxic drugs for treating any of the aforementioned forms of cancer induce many side effects due to non-specific distribution in the whole body, affecting other organs with only a few percent reaching the target organ “skin”. The skin is an interesting alternative route for drug administration^{[2]}, particularly when the tissue itself is the disease site^{[2-4]}. Consequently, there is an increasing clinical demand for local therapies for skin cancer that ensure cutaneous tumor localization of drugs while limiting its systemic absorption.

Bexarotene (BEX) is a lipophilic, third generation retinoid that selectively activates the retinoid X receptor (RXR) subtypes of retinoid receptors. Once activated, these receptors function as transcription factors that regulate the gene expression, controlling cellular differentiation and proliferation^{[5]}. It exhibits anti-tumor activities by inhibiting cell growth, and promoting apoptosis in a variety of tumor cell lines^{[6-8]}. It is clinically prescribed as a long-term treatment to suppress the progression of cutaneous T-cell lymphoma (CTL)^{[9-11]}. It is extremely effective against non-small cell lung cancer (NSCLC) and has shown promising activity against ER-negative breast cancer and prostate cancer^{[3, 12]}. However, BEX has an extremely low aqueous solubility (˜10 μM), resulting in its poor has led to poor bioavailability. This has hampered its pharmaceutical development and clinical application. Moreover, BEX causes side effects such as hypothyroidism and elevation of serum triglycerides (Hypertriglyceridemia)^{[13, 14]} which can lead to pancreatitis, while increasing the risk for cardiovascular disease. Accordingly, CTCL patients are prescribed a combined regimen of BEX, a statin and tetraiodothyronine as thyroid hormone replacement. This complex regimen increases cost, reduces compliance, and could elicit additional side effects, especially from statins. Therefore, developing BEX formulations for localized therapies without elevating the triglyceride levels is a high clinical priority among dermatologists, especially for patients with CTLs.

Vorinostat (VOR) is another drug approved in the United States for the treatment of progressive, persistent or recurrent CTCLs^{[15]}. It is an HDAC inhibitor (HDACI) that is rapidly metabolized in vivo by both glucuronidation and oxidative degradation of the alkyl chain to 4-anilino-4- oxobutanoic acid^{[16]}. In addition to its poor aqueous solubility, vorinostat suffers from low cell permeability and can induce drug resistance^{[17]}. It is primarily available as an oral tablet which can cause several side effects including hyperglycemia, thrombocytopenia, alopecia, taste disorders, and the more serious adverse effects such as pulmonary embolism and anemia, with a poor clinical outcome^{[18]}.

The inventors aimed to design a topical delivery platform that provides cutaneous localization of both drugs at therapeutically relevant concentrations while minimizing systemic absorption. The inventors examined the feasibility of hyaluronic acid-conjugation to allow anti-cancer drugs to cross the skin barrier for advanced topical treatment of cutaneous malignancies.

HA was chosen based on its unique properties being a non-toxic, non-immunogenic biocompatible, and biodegradable natural polymer. Use of HA conjugates for topical treatment of skin cancers has not previously been reported.

Described herein is the conjugation of BEX and VOR to HA via an ester linker. The synthesis and physico-chemical characterization is provided. The inventors tested the toxicity of these conjugates against human cutaneous cancer cell lines and demonstrated their safety with primary normal human keratinocytes and primary human fibroblasts. The inventors demonstrated the ex-vivo skin penetration of conjugates with human skin explants and demonstrate their internalization by cancer cells. The biodistribution of the conjugates in the skin layers was also assessed.

Materials

Bexarotene (BEX) and Vorinostat (VOR) were purchased from T.C.I (St. Louis, Mo., USA). N-(3-Dimethylaminopropyl)-N′-ethylcarbodiimide hydrochloride (EDC), and 4 (dimethylamino) pyridine (DMAP) were purchased from sigma-Adlrich (St. Louis, Mo., USA). Alexa Fluor 647 and Alexafluor 488 were obtained from Thermofisher Scientific Hyaluronic acid (HA) of 50 kDa MW was purchased from Creative PEGWorks (Winston Salem, N.C., USA). AlamarBlue (AB) was from ThermoFisher Scientific, Hoechst was purchased from Invitrogen Life Technologies (San Diego, Calif., USA). Human cutaneous T cell lymphoma (HUT 78) and human squamous cell carcinoma (A431) cell lines, Dulbecco's Modified Eagle's medium (DMEM) and Iscove's Modified Dulbecco's Medium (IMDM) were acquired from ATCC (USA). Primary human keratinocytes were provided by the Cutaneous Biology Center at Massachusetts General Hospital and primary human fibroblasts were purchased from Angioproteomie, USA. Fetal bovine serum (FBS), phosphate buffered saline (PBS), 0.25% trypsin, penicillin/streptomycin, and Nunc Lab-Tek 8-chambered coverglasses were purchased from Thermo Scientific. Cell culture flasks and microplates were obtained from Corning (NY, USA). Sephadex G-25 PD-10 columns were obtained from GE Healthcare Life Sciences (Piscataway, N.J., USA), and microcentrifuge filter tubes were purchased from EMD Millipore (Billerica, Mass., USA). HPLC grade Acetonitrile, trifluoroacetic acid and all other chemicals used were obtained from Fisher Scientific and at the highest possible grade available.

Methods

Synthesis of HA-BEX, and HA-VOR

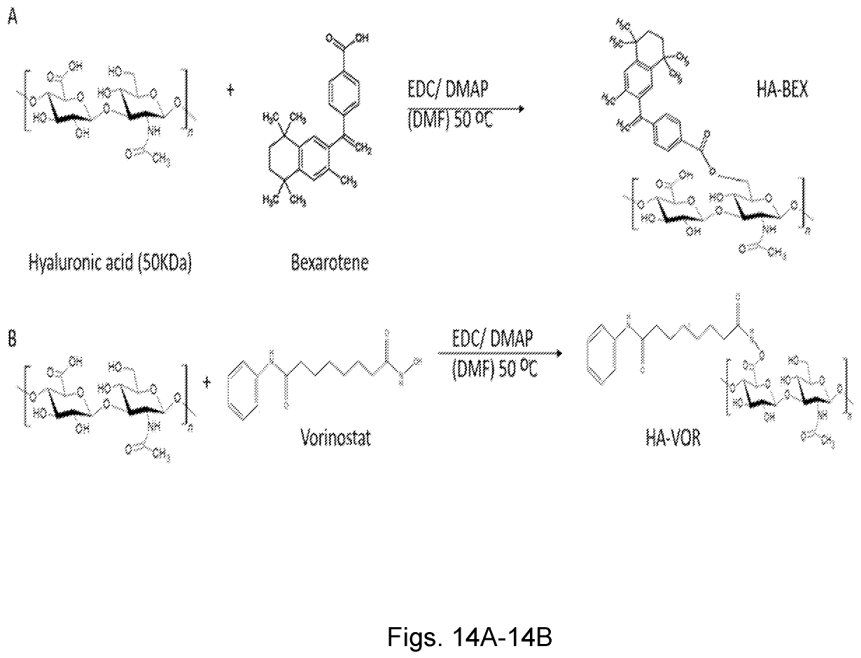

BEX and VOR were conjugated to HA via ester bond formation (FIGS. 14A-14B). For the synthesis of HA-BEX, 7 mg of Bex (0.052 mmol) were dissolved in 200 ul DMSO followed by the addition of 20 mg EDC (0.104 mmol) and 6 mg (0.049 mmol) of DMAP dissolved in DMSO. The mixture was stirred for 1 hour at room temperature then was added to 40 mg of 50 kDa MW HA (0.104 mmol of HA acid units) dissolved in 2 mL DMF under stirring and slight heating (50° C.). The reaction was allowed to proceed for 48 hr. The product was purified by size exclusion chromatography through Sephadex G-25 PD-10 desalting columns equilibrated in MilliQ water. The product was further concentrated using centrifugal filter tubes (MW cutoff 3000 NMWL) for a minimum of three runs, each at 4500 rpm for 15 min followed by washing with MilliQ water. The concentrated product was then lyophilized (SP Scientific Freezemobile) for storage and further characterization.

For the synthesis of HA-VOR, 20 mg of HA (0.052 mmol HA units) were dissolved in 1 ml DMF at 50° C., 30 mg EDC and 6 mg DMAP were added. The mixture was stirred for 15 minutes followed by the addition of 7 mg (0.026 mmol) VOR dissolved in 200 ul DMSO. The reaction was left for 48 hours. The product was purified by repeated precipitation and washing using a mixture of ethanol and PBS until no free drug is detected in the supernatant. The precipitate was then lyophilized for storage and characterization.

BEX-HA and VOR-HA conjugates were characterized by ¹H NMR, FTIR, DLS and TEM. Drug loading was determined via UV-Vis measurements performed using plate reader (spectra imax3). The λₘₐₓ used for quantifying each drug was 260 nm for BEX and 242 nm for VOR respectively. HA at the same concentration range was used as a blank. Chemical structures of HA conjugates were verified with ¹H NMR. The conjugates were dissolved in D20 at 5-7 mg/mL and analyzed with a Varian VNMRS (600 MHz) spectrometer. Data were processed with the software Mestrenova (Bruker GmbH, Karlsruhe, Germany).

The physical structure of the conjugates was visualized with transmission electron microscopy (Hitachi 7800). Samples were applied directly onto carbon film on 200 mesh copper grids. Any excess sample was removed carefully by capillary action, and the grids were immediately stained with one drop of uranyl acetate for 30 s. The size and zeta potential of the conjugates were determined by DLS (Malvern Zetasizer NanoZS instrument (Malvern Instruments Ltd., Worcestershire, UK), equipped with a 532 nm laser at a fixed scattering angle of 173°. The conjugates were diluted in PBS at a concentration of 0.1- 1 mg/ml, sonicated for 10 min, filtered through a 0.45 m cellulose membrane filter, and then analyzed. Z-potential measurements were made using disposable folded capillary cells. All measurements were performed in triplicates.

High Performance Liquid Chromatography (HPLC)

A reversed phase HPLC method (Agilent, USA) was adopted for the quantitative determination of BEX and VOR in the skin samples. The measurements were performed using an RP C-18 LiChrospher analytical column (125×4.0 mm) (Scharlab S.L.), with a flow rate of 1 mL/min, an injection volume of 10 μL and a gradient elution method with a mobile phase of H₂O (0.1% TFA)/ACN (60/40). The detection wavelengths were, =242 and 260 nm and the retention times were 5 min for VOR and 11 min for BEX respectively. The calibration curves were linear within the concentration range of 0.2-100 ug/mL.

Synthesis of Fluorescent HA-BEX and HA-VOR

100 mg of HA was dissolved in 10 ml phosphate buffer pH 5.5, followed by the addition of 1.5 mg EDC dissolved in 30 ul DMSO. Later, 1 mg of Alexaflour 647 hydrazide was added to the mixture. A similar procedure was followed for the synthesis of HA labelled with Alexaflour 488 hydrazide as well. The reaction was allowed to proceed under gentle stirring in the dark overnight. The product was purified by repeated precipitation and washing steps using ethanol/PBS mixture until no fluorescence was detected in the supernatant. The fluorescent HA was then lyophilized. Fluorescent HA-BEX was prepared following the same procedure used for the synthesis of HA-BEX using HA labelled with Alexaflour 647. Similarly, fluorescent HA-VOR was prepared in the same manner as HA-VOR using HA labeled with Alexaflour 488.

Release of BEX or VOR from HA Conjugates

Drug release of BEX or VOR from its conjugates was studied using Slide-A-Lyzer MINI Dialysis Devices of 10,000 MWCO (Life Technologies, Grand Island, N.Y.). HA-conjugates were placed in dialysis cups with either PBS buffer (pH 7.4) or pH 5.5 (adjusted by 0.1 N HCL) and were inserted into centrifuge tubes containing 15 mL of the same buffer. Dialysis devices were placed at 37° C. and allowed to shake at 100 RPM. At indicated time points, concentrations of BEX or VOR in the buffer surrounding the dialysis cup were determined by HPLC.

In Vitro Cell Toxicity and Synergy Determination

A431 cells were cultured in DMEM media supplemented with 10% FBS, 1% Pen Strep. The cells were passaged 3-4 times prior to setting up the assay. HuT 78 cells were culture in IMDM supplemented with 20% FBS. Cells were cultured in a humidified incubator maintained at 37° C. and 5% C02 (Thermo scientific, USA). A431 Cells (5×10³ in 100 ul in complete DMEM) were seeded in 96 well plates and allowed to adhere overnight. The next day, cell culture media was aspirated and replaced with the different treatments. The cells were incubated with the drug solutions for 48 hr after which the treatment was aspirated and replaced with 10% alamar blue (AB) solution in complete DMEM and incubated for an additional 3.5 h in the dark at 37° C. Similarly, HUT 78 cells, (5×10³ cells in 50 ul IMDM) were seeded in U bottom well plates and treatments were added to a final volume of 100 ul in each well. After 48 h, the plates were centrifuged to collect the cell suspension at the plate bottom and 100 ul of IMDM containing 10% AB was added to each well. The plates were then incubated in the dark for 4 h. Finally, cellular viability was assessed by reading the florescence of the solution from each well at λex=560 nm and λem=590 nm. Spectra imax3). Baseline fluorescence levels from wells without cells were subtracted from each data (Dose response curves were fit to each drug and/or polymer drug conjugate with the median effect model^{[28]}. For combination treatment, synergy was quantified with the combination index^{[29]}

Uptake of HA-BEX and HA-VOR by A431 and HUT78 Cells.

The uptake of fluorescently labelled conjugates was studied by confocal microscopy. For A431 cells, 5×10³ cells were plated in individual chambers of Nunc™ Lab-Tek™ II Chamber Slide™ System (Thermo Fischer Scientific) and allowed to adhere overnight. The media was then aspirated and replaced with 200 ul of fluorescently labelled HA-BEX or HA-VOR, followed by incubation for 4 hr at 37° C. in the incubator. The treatments in the wells were completely aspirated and cells were washed 2 times with PBS before fixing with 4% paraformaldehyde. The fixed cells were mounted using Fluroshield® to stain for DAPI (Ex/EM 340/488 nm) and were imaged using confocal microscopy (Upright Zeiss LSM 710 NLO ready, Germany). For HUT 78 cells, 5×10³ cells were plated in U-shaped bottom 96 well plate and treated with the florescent conjugates for 4 h after which the plate was spun down the treatment was aspirated, the cells were washed with PBS spun down, fixed with 4% paraformaldehyde and the nuclei were stained with Hoechst 33342 for 15 min, washed with PBS, transferred to a glass slide and imaged by CLSM. Hoechst 33342 and Alexaflour 647, were imaged using excitation/emissions of 638/756 and 493/517 nm, respectively. The uptake was also quantified by flow cytometry ((BD LSR Analyzer II, CA, USA) after 1 and 4 h.

Cell Viability Study in Keratinocytes and Fibroblasts

Primary human fibroblast (seeded at density 5,000 cells/well) and primary human keratinocytes (at density 2,000 cells/well) were used for the cytotoxicity studies in vitro. The culture media used for the keratinocytes was keratinocyte serum free medium supplemented with L-glutamine, EGF and BPE while that for fibroblasts was Dulbecco's Modified Eagle's Medium-high glucose supplemented with 2% penicillin/streptomycin and 5% FBS. Cells were incubated in a humidified incubator under 5% CO₂ at 37° C. After 24 h, 50 μL of the media were withdrawn and replaced by 50 ul of increasing concentrations of each treatment reaching a final volume of 100 μL in the well. Cells were incubated for 48 h under standard cell culture conditions after which cell viability was assessed by AB assay as previously described.

Visualization of Dermal Penetration of HA-BEX and HA-VOR

Porcine skin (Lampire Biological Laboratories, Pipersville, Pa.) was kept at −80° C. and thawed immediately prior to use. Skin discs were punched out to fit the area of the diffusion cells, followed by trimming of any surface hair using clippers. The skin samples were clamped between the two compartments of Franz diffusion cells (area 2 cm²) with the stratum corneum facing the donor compartment. The acceptor component of the cell was filled with PBS and equipped with a magnetic stirrer bar. 200 uL of each of fluorescently labeled HA-BEX or HA-VOR (20 mg/mL) was placed on top of the skin, ensuring full coverage. The cell was placed on a stirrer plate at 37° C. for 24 hours, then the cell was dismantled, the skin surface was washed with PBS and wiped with cotton swap to ensure complete removal of any residual treatment. The skin area in contact with the treatment was punched out and subsequently embedded in OCT using a cryomold and then snap-frozen in isopentane cooled in liquid nitrogen followed by mounting into a cryotome. Samples of 20 um slices were taken using a cryostat (Leica CM1950, Leica, Germany). The slices of skin were placed on superfrost glass slides. (Fisher Scientific, Pittsburgh, Pa., USA) and were fixed in formaldehyde 4% for 15 min and mounted with Flouroshield with DAPI for staining the nuclei then adding cover slip^{[30]}. The slides were imaged using a confocal microscope (Upright Zeiss LSM 710 NLO) with a 10× air objective. Excitation and emission wavelengths for (i) DAPI were 353 and 465 nm and (ii) Alexa488 were 493 and 517 nm while for Alexa 647 were 638 and 756 nm respectively. Images were acquired using an Axiocam 506 camera. All images acquired with the same objective used the same exposure time, lamp intensity and grey pixel setting in order to allow accurate comparison of signals. Images were acquired and processed using Zen 3.1 pro software.

Quantification of BEX and VOR in the Skin

The permeation of HA-BEX and HA-VOR conjugates (500 μL of 20 mg/mL aqueous dispersion) in the skin was studied using porcine skin over 24 h in Franz diffusion cells. After 24 h, the skin was washed twice with PBS. The SC and epidermis were separated from the dermis. Each of the two layers was cut into small pieces and added to 4 mL of 1:1 mixture of ethanol and PBS. Extraction was performed by sonication for 2 hr followed by stirring for 24 h. The samples were then centrifuged and the supernatant was filtered (0.45 μm) into HPLC vials for analysis. Samples were measured repeatedly (n=3).

Visualization of Penetration on Human Skin Explant

Human skin samples were obtained from healthy women following plastic surgery after written informed consent (Cutaneous Biology Center, Massachusetts General Hospital, USA). The skin samples were placed with the dermal side down onto membranes (8.0-mm polycarbonate membranes, Transwell Permeable Supports, Costar, Pittsburgh, Pa.) that were in contact with the growth medium RPMI, containing 10% human serum (Gemini Bio Products, West Sacramento, Calif.) and antibiotics/antimycotics. The edges of the skin were sealed with semisoft agar, and the apical surface was kept in contact with air. Human skin samples were acquired following the procedures established by MGH. 50 μl of either florescent HA-BEX, florescent HA-VOR or a mixture of both at a concentration of 20 mg/ml was applied to the skin surface followed by incubation for 24 h at 37° C. The skin samples were then snap frozen in OCT and mounted on cryotome. 20 uM slices were obtained, mounted with Flouroshield with DAPI and examined by CLSM as previously described for the porcine skin.

Results and Discussion

Synthesis and Characterization of HA-BEX and HA-VOR

The inventors conjugated two poorly water-soluble drugs BEX and VOR to a water-soluble HA carrier via an ester bond to improve their cutaneous penetration and bioavailability via topical application. to mediate a localized treatment of cutaneous malignancies. HA offers the advantage of bearing carboxylic acid as well as hydroxyl groups, thus allowing the formation of ester bond with —OH or —COOH bearing drugs respectively. Esterification reaction was mediated by EDC, a carbodiimide coupling reagent, and DMAP as a catalyst (FIGS. 14A-14B).

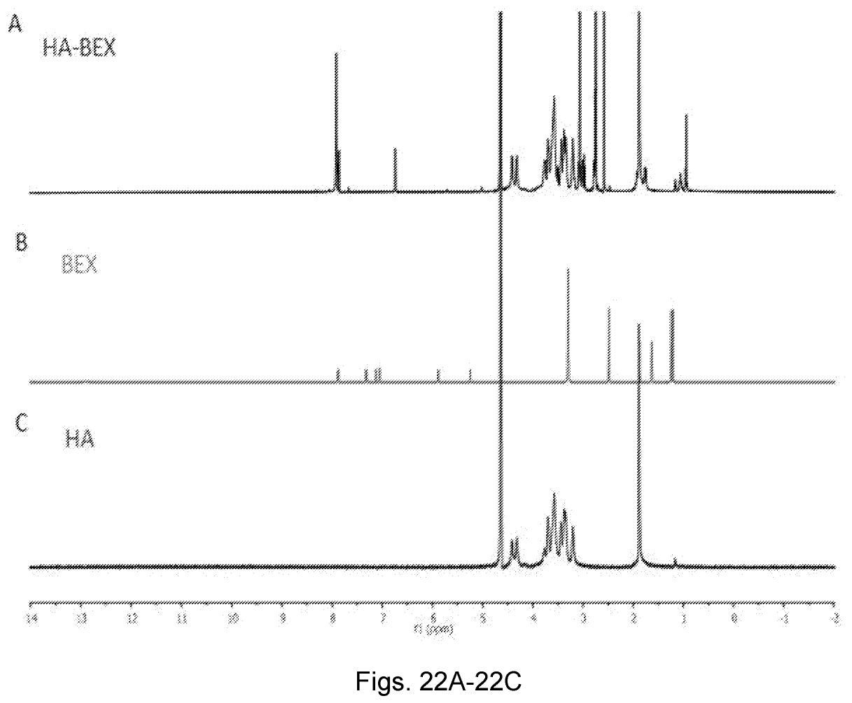

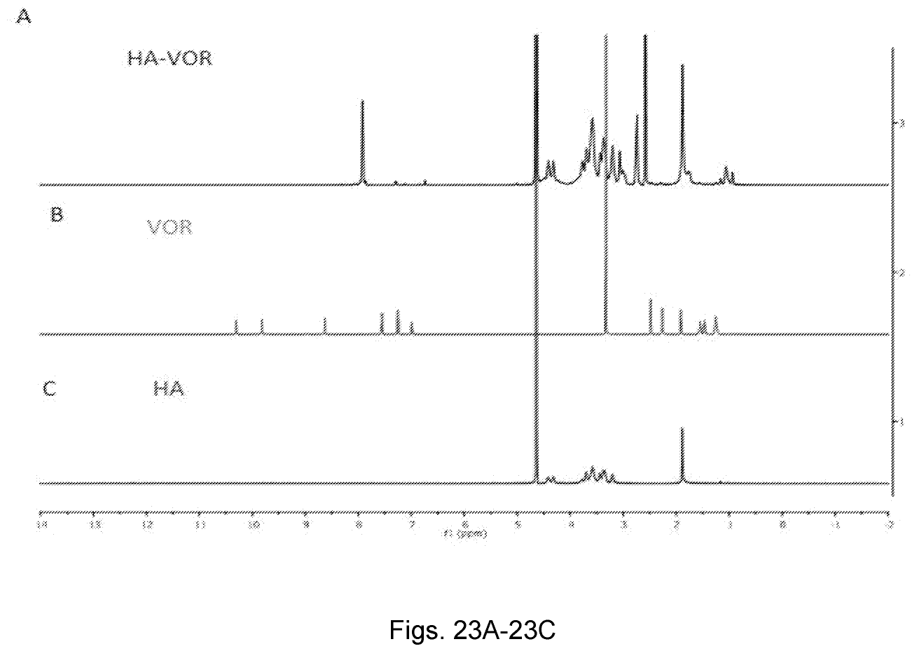

50 kDa HA was selected as a backbone. Conjugates were characterized using a number of techniques to confirm identity, purity, and quantify the total drug loaded. ¹H NMR spectroscopy was used to confirm BEX conjugation to HA (FIGS. 22A-22C). HA and HA-BEX showed a peak, characteristic of the polymer at 1.88 ppm (—CH3-g of the acetamido moiety of N-acetyl-D-glucosamine). The characteristic BEX peaks at 7.96, 7.35 and 7.14 ppm that correspond to its benzene moiety appeared in the HA-BEX spectrum, confirming the drug's successful conjugation. Peak integration indicated a BEX molar content of ˜9.1 mol %. For HA-VOR, ¹H NMR also showed the characteristic peak for HA at 1.88 ppm and characteristic peaks for VOR at 0.93-2.45 ppm that represents its aliphatic protons. Compared with the ¹H-NMR spectrum (FIG. 23B) of VOR the peak at 10.4 ppm related to the hydroxyl proton of VOR disappeared completely confirming its involvement in ester bond formation with the carboxylic group of HA. (FIG. 23A). However, the aromatic protons of VOR could not be clearly detected which can be due to its presence in the core of self-assembled aggregates of the HA conjugate. A similar observation was previously reported for HA-DOX^{[25]}. The molar content of VOR in the HA-VOR conjugate was 4.8 mol % as determined by ¹H NMR and this was further confirmed by UV spectrophotometry. The inventors optimized the synthetic methodology to improve the consistency yielding the desired products with high purity. The higher drug loading achieved in case of HA-BEX can be attributed to the fact that hydroxyl groups are more abundant than carboxyl group providing better reaction opportunities.

Drug loading was also confirmed by UV-Vis analysis. The inventors determined the total drug loading of the conjugates (0.1 mg/mL in MilliQ H2O/ethanol (20:80) at 260 nm and 242 nm for BEX and VOR respectively. Both conjugates preserved the characteristic maximum absorbance of the parent drugs. Drug loading of about 7.5 wt. % and 3.25% could be achieved for BEX and VOR respectively. The conjugates were soluble with no visible macroscopic aggregates.

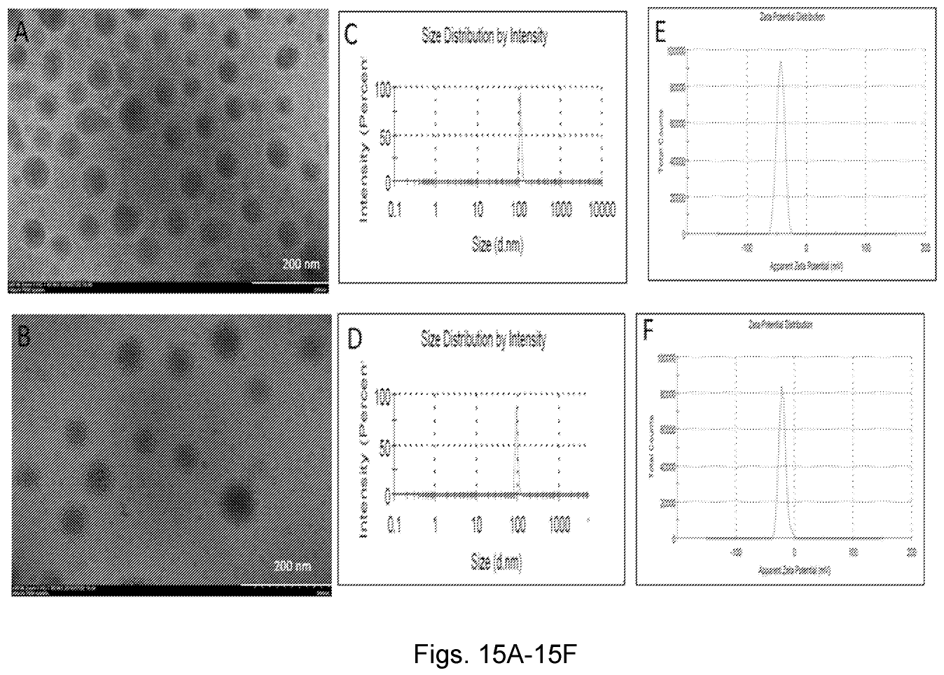

The size of HA-BEX and HA-VOR conjugates was measured with DLS and morphology was visualized using transmission electron microscopy (TEM). TEM micrographs confirmed the spherical aspect of both conjugates suggesting the formation of self-assembled structures with geometrical sizes in the range of 50-100 nm. This was further confirmed with DLS measurements where HA-BEX and HA-VOR exhibited hydrodynamic diameters of 100±9.7 nm and 90.5±10 nm respectively and zeta potential values of −43.6±3.2 and −23±3.1 mV respectively (FIGS. 15A-15F).

In Vitro Cytotoxicity and Cellular Uptake

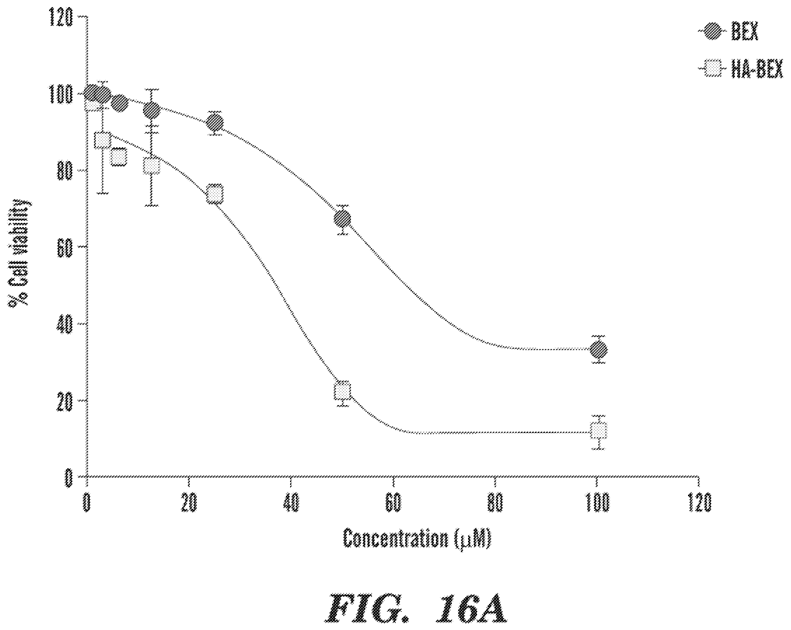

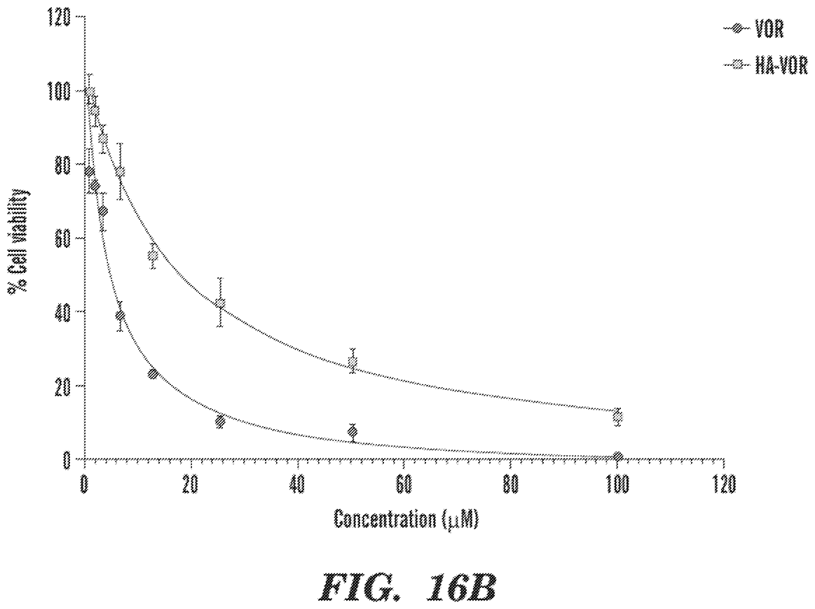

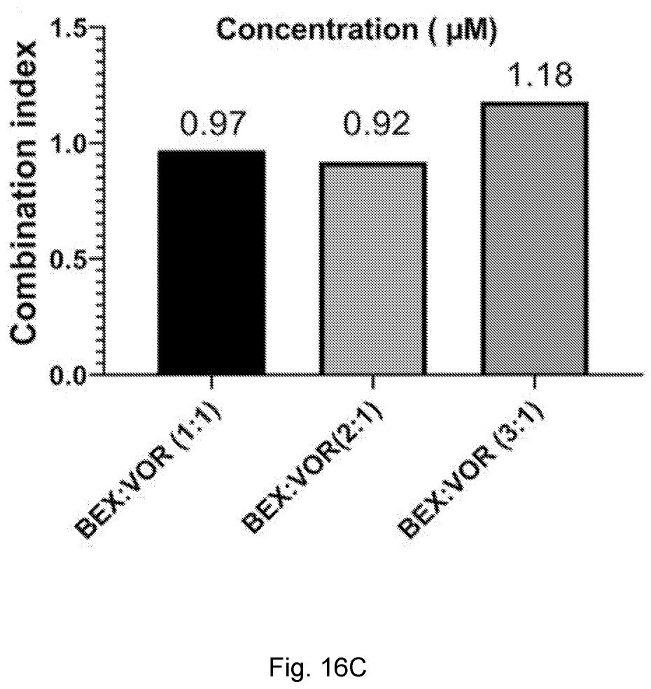

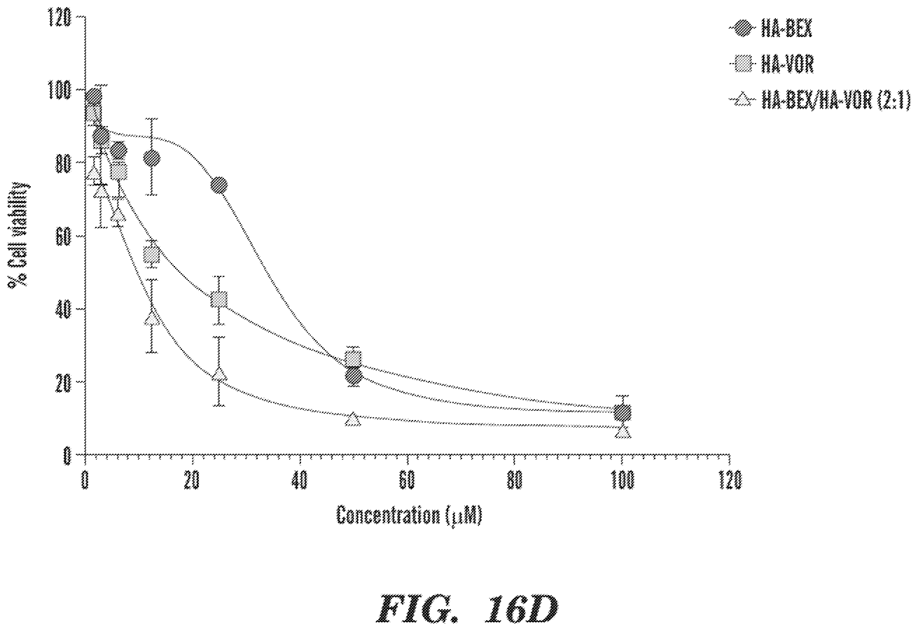

The anticancer efficacy of BEX, VOR, the individual HA-conjugates and their combinations was tested on human squamous cells (A431) at increasing concentrations for a period of 48 h. (FIGS. 16A-16D). The activity of HA-BEX conjugate (IC50=34.2 μM) was enhanced compared to that of free BEX (IC50=60.32 μM). On the other hand, conjugation to HA reduced the activity of VOR (IC50=17.9 μM) compared to the free drug (IC50=5.46 μM).

The inventors also studied the effect of combining BEX and VOR at different ratios on the viability of A431 cells. The Combination Index (CI) method was adopted to characterize the combination potencies. Synergism, additivity and antagonism were indicated by CI values less than 1, equal to 1 and greater than 1, respectively. Bex and VOR showed synergism in principle at ratios of 1:1, 2:1 but not 3:1 (FIG. 16C). The extent of synergy, however, was modest. The inventors tested the effect of combining both conjugates at ratio of 2:1. The combined conjugates were more effective in inhibiting cell growth compared to the individual conjugates (FIG. 16D). The IC50 of HA-BEX in the combination is 10.98 μM and that of HA-VOR is 5.54 μM.

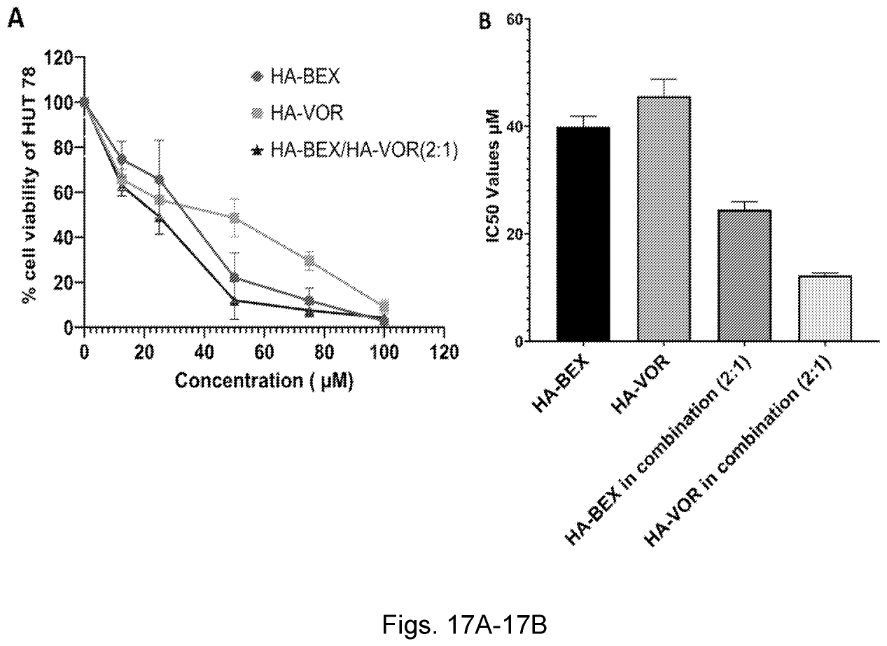

The inventors also examined the effect of HA-BEX and HA-VOR drug combinations on cutaneous T-cell lymphoma cell line (HUT 78). At all concentrations, the combined conjugates showed better cell inhibitory activity compared to the individual conjugates (FIG. 17A). A reduction in the IC50 of both conjugates was observed at a combined ratio of 2:1 (FIG. 17B).

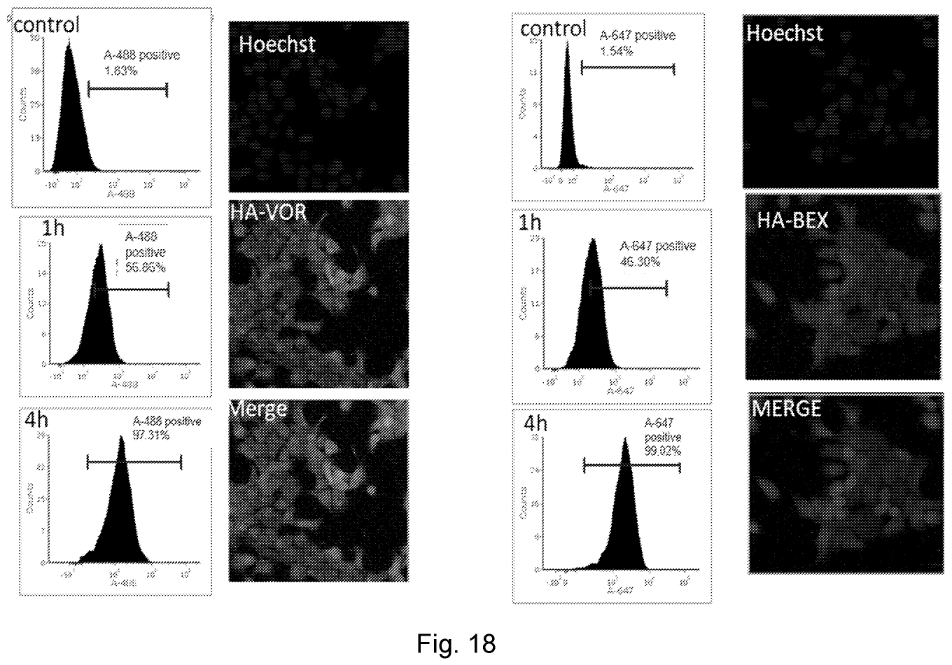

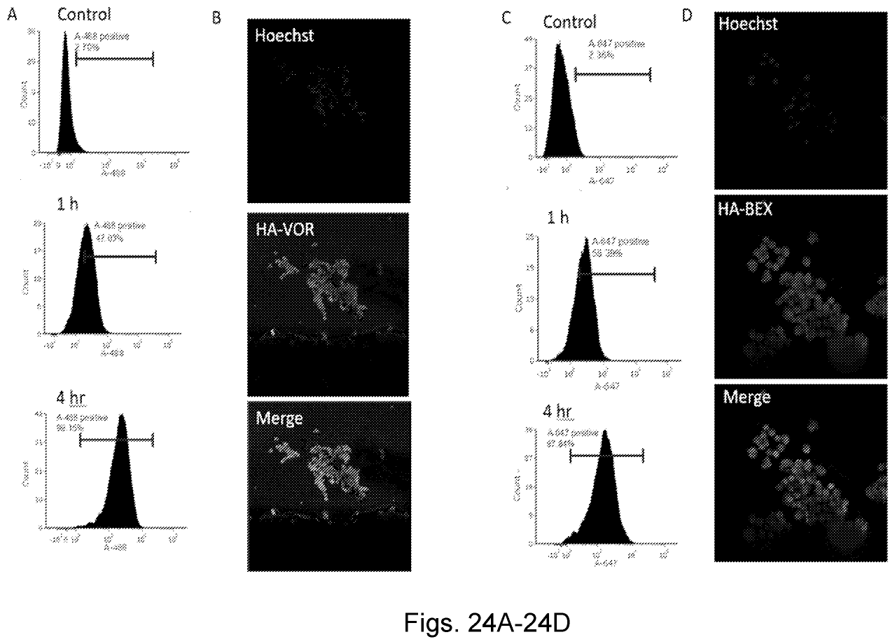

Efficient interaction of polymer-drug conjugate with the target cancer cells and subsequent uptake is critical for enhanced delivery and activity. The inventors evaluated this by studying the uptake of the conjugates by A431 and HUT78 cells. CLSM images demonstrate that HA-conjugates are internalized by both A431 (FIG. 18), and HUT 78 (FIGS. 24A-24D) cells efficiently within 4 h of the incubation. This was also confirmed by flow cytometry showing an increase in florescence intensity following an increase in the treatment time from 1 hr to 4 hr (FIG. 18 and FIGS. 24A-24D). The increase in fluorescence within the cell with time indicated an effective endocytic intracellular delivery.

Effect on Human Dermalfibroblasts and Keratinocytes

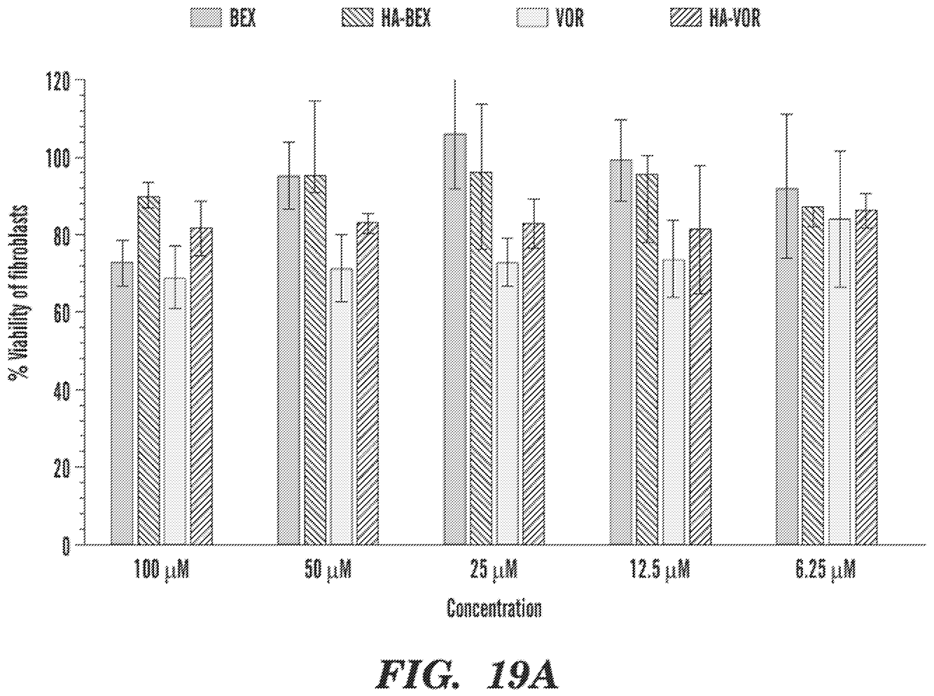

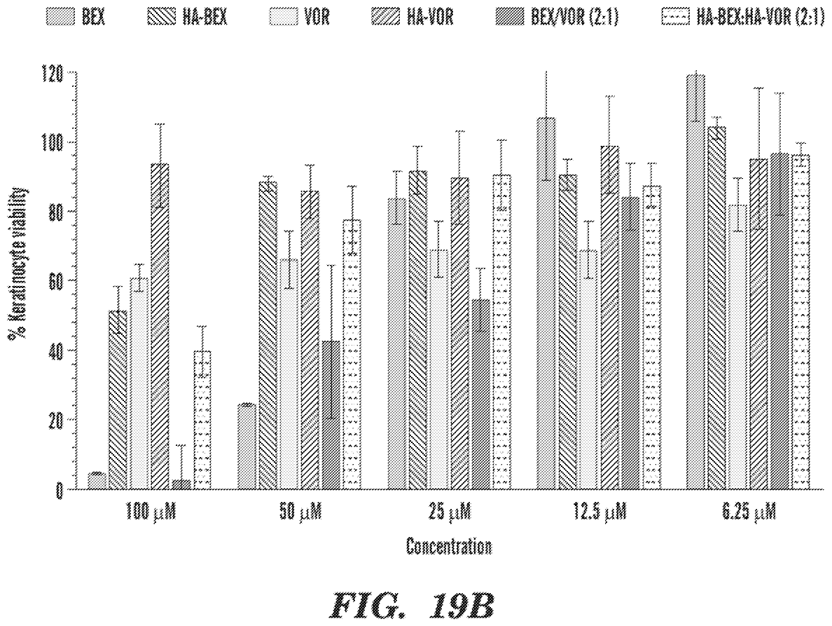

The effect of the BEX, VOR and their conjugates on the viability of primary human fibroblasts and keratinocytes was tested. In vitro monolayers of dermal fibroblasts have been used in different in vitro assays to predict the adverse effects of topically applied substances^{[31, 32]}. Treatment of human dermal fibroblasts with increasing concentrations of the Bex, VOR and their HA-conjugates did not significantly affect the cell viability relative to the untreated control, indicating the absence of skin toxicity. This confirms the safety of using HA-BEX and HA-VOR conjugates for dermal applications (FIG. 19A). The inventors also evaluated cell viability of primary human keratinocytes in response to the same concentrations of BEX, VOR and their HA-conjugates tested on the cancer cells (FIG. 19B). Interestingly, both conjugates exhibited lower inhibitory effect towards keratinocytes than the free drugs used at the same concentrations.

Ex Vivo Skin Penetration

HA-BEX and HA-VOR penetrated into porcine skin in vitro over a period of 24 h (FIGS. 24A-24D and data not shown). For both conjugates, the photo micrographs illustrate the ability of the conjugates to cross the stratum corneum and deliver the attached drugs to deep skin layers. Topical application of free aqueous Alexa 647 or Alexa488 (data not shown), resulted in surface accumulation of the dyes with no penetration beyond. This indicates that the fluorescent signals detected after applying the fluorescently labeled conjugates is due to the enhanced penetration of the HA conjugates.

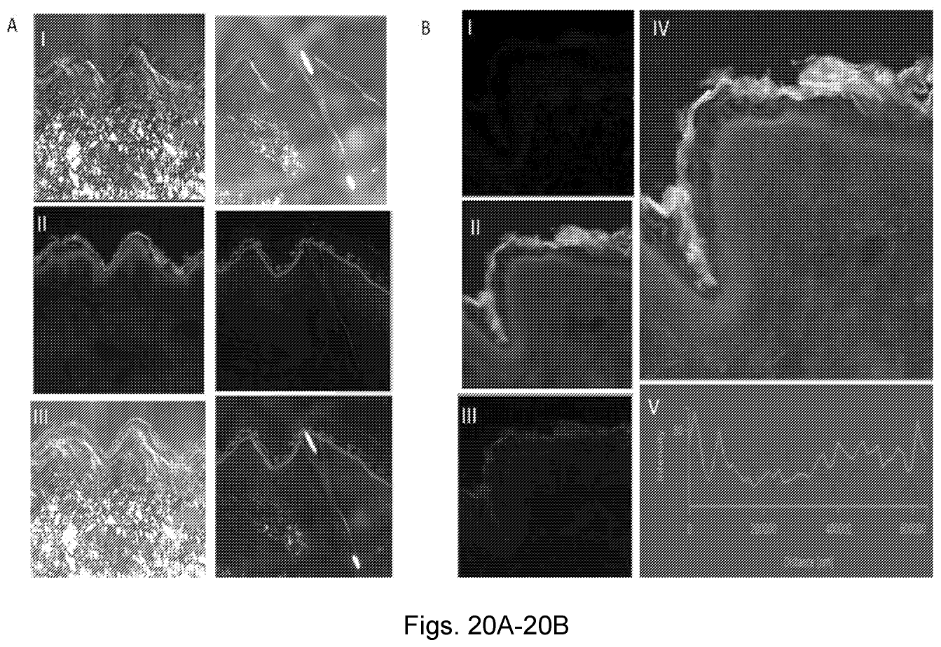

Healthy human skin explant harvested during plastic surgery were kept alive using a well-established protocol^{[33]}. The florescent conjugates were applied to the skin either individually or combined together for a period of 24 hr after which the skin was snap frozen, processed and imaged (FIGS. 20A-20B). The human skin images confirmed the observation noticed for the porcine skin. The presence of a strong florescence signal across the epidermis, dermis and the entire depth of a hair follicle proved the ability of HA-BEX and HA-VOR to penetrate deep inside the skin and accumulate at sites where cancer cells invade and multiply.

Quantification of Skin Retention and Penetration

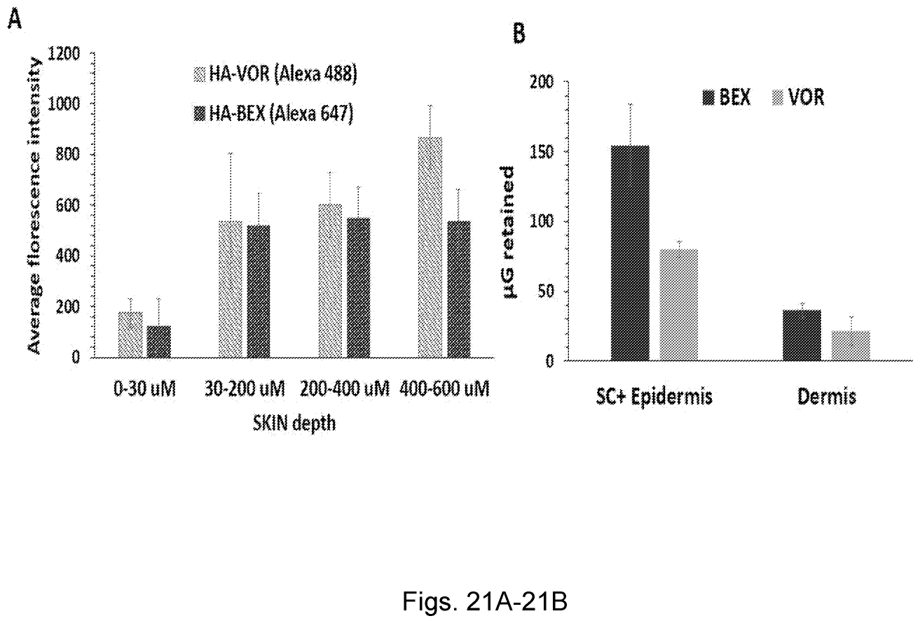

The inventors quantified fluorescence intensity in various skin layers by ZEN 3.1 software after the application of the fluorescent HA-conjugates. The fluorescence intensity was plotted versus the skin depth (FIG. 21A). The uppermost 30 μm of the skin represents the SC, the next 30-200 μm below represents the viable epidermis, and the remaining portion of the skin represents the dermis. A higher amount of the conjugates accumulated in the epidermal layers compared to the dermis proven by the higher fluorescence intensity detected. Moreover, fluorescence was detected up to a depth of 600 μm. Furthermore, the permeation and distribution of both BEX and VOR in the different skin layers was studied in order to evaluate the ability of the elaborated HA-conjugates to deliver the anticancer agents to the desired site of action and also to estimate the extent of systemic absorption which is an important safety aspect in the development of topical drug delivery systems.

An HPLC method was developed to quantify the amount of BEX and VOR in the epidermis and dermis. FIG. 21B represents the amounts of BEX and VOR retained per unit surface area of the stratum corneum and the epidermis as well as the dermis. We also evaluated the aliquots from the receptor compartment of the Franz diffusion cells at 24 h of permeation. No drug was detected in the receptor compartment suggesting that the drugs are retained in the skin with no tendency to penetrate further. or that the amount of drugs that fully permeates up to the receptor chamber is below the detection limit of the equipment (<0.2 ug/ml). The successful permeation of conjugates across the SC and the deeper localization could be attributed to the increased hydration of the surface layers which in turn opens up the barrier by loosening the dense, closely packed cells, thereby increasing the permeability^{[34]}. The increased hydration of the skin's surface layers also facilitates cargo retention within the more hydrated epidermal layers, thereby decreasing drug diffusion into the lower skin layers and access to the systemic circulation^{[31]}. The dermal accumulation of HA-Conjugates can also be attributed to the presence of HA receptors that support enhanced binding and localization.

Discussion

Described herein is an effective topical therapy for skin cancer by conjugating two chemotherapeutic agents (BEX and VOR) to HA. The conjugates exhibited synergistic effect against human SCC and CTCL cell lines, promoted drug permeability and localization within the target skin layers, with minimal effect on the viability of normal skin cells. Such topical therapies for cutaneous malignancies, provides an alternative to systemic administration, thereby avoiding serious side effects.

### REFERENCES

[1] R. L. Siegel, K. D. Miller, A. Jemal, CA Cancer J Clin 2015, 65, 5.
[2] M. R. Prausnitz, R. Langer, Nat Biotechnol 2008, 26, 1261.
[3] Y. Chen, P. Quan, X. Liu, M. Wang, L. Fang, Asian Journal of Pharmaceutical Sciences 2014, 9, 51.
[4] K. Higaki, M. Asai, T. Suyama, K. Nakayama, K. Ogawara, T. Kimura, Int J Pharm 2002, 239, 129.
[5] P. Germain, P. Chambon, G. Eichele, R. M. Evans, M. A. Lazar, M. Leid, A. R. De Lera, R. Lotan, D. J. Mangelsdorf, H. Gronemeyer, Pharmacol Rev 2006, 58, 760.
[6] L. T. Farol, K. B. Hymes, Expert Rev Anticancer Ther 2004, 4, 180.
[7] L. S. Qi, Y. Y. Guo, J. J. Luan, D. R. Zhang, Z. X. Zhao, Y. X. Luan, Journal of Materials Chemistry B 2014, 2, 8361.
[8] S. K. Misra, G. Ghoshal, M. R. Gartia, Z. Wu, A. K. De, M. Ye, C. R. Bromfield, E. M. Williams, K. Singh, K. V. Tangella, L. Rund, K. Schulten, L. B. Schook, P. S. Ray, E. C. Burdette, D. Pan, ACS Nano 2015, 9, 10695.
[9] N. Mehta, A. S. Wayne, Y. H. Kim, G. A. Hale, C. S. Alvarado, P. Myskowski, E. S. Jaffe, K. J. Busam, M. Pulitzer, J. Zwerner, S. Horwitz, Clin Lymphoma Myeloma Leuk 2012, 12, 20.
[10] R. A. Abbott, S. J. Whittaker, S. L. Morris, R. Russell-Jones, T. Hung, S. J. Bashir, J. J. Scarisbrick, Br J Dermatol 2009, 160, 1299.
[11] M. Duvic, A. G. Martin, Y. Kim, E. Olsen, G. S. Wood, C. A. Crowley, R. C. Yocum, G. Worldwide Bexarotene Study, Arch Dermatol 2001, 137, 581.
[12] Y. Li, Y. Zhang, J. Hill, H. T. Kim, Q. Shen, R. P. Bissonnette, W. W. Lamph, P. H. Brown, Br J Cancer 2008, 98, 1380.
[13] C. Assaf, M. Bagot, R. Dummer, M. Duvic, R. Gniadecki, R. Knobler, A. Ranki, P. Schwandt, S. Whittaker, Br J Dermatol 2006, 155, 261.
[14] R. E. Hurst, Curr Opin Investig Drugs 2000, 1, 514.
[15] M. Duvic, E. A. Olsen, D. Breneman, T. R. Pacheco, S. Parker, E. C. Vonderheid, R. Abuav, J. L. Ricker, S. Rizvi, C. Chen, K. Boileau, A. Gunchenko, C. Sanz-Rodriguez, L. J. Geskin, Clin Lymphoma Myeloma 2009, 9, 412.
[16] L. Du, D. G. Musson, A. Q. Wang, J Pharm Biomed Anal 2006, 42, 556.
[17] E. A. Olsen, Y. H. Kim, T. M. Kuzel, T. R. Pacheco, F. M. Foss, S. Parker, S. R. Frankel, C. Chen, J. L. Ricker, J. M. Arduino, M. Duvic, J Clin Oncol 2007, 25, 3109.
[18] M. Flipo, J. Charton, A. Hocine, S. Dassonneville, B. Deprez, R. Deprez-Poulain, J Med Chem 2009, 52, 6790.
[19] M. T. Epping, L. Wang, J. A. Plumb, M. Lieb, H. Gronemeyer, R. Brown, R. Bernards, Proc Natl Acad Sci USA 2007, 104, 17777.
[20] F. F. Ferrara, F. Fazi, A. Bianchini, F. Padula, V. Gelmetti, S. Minucci, M. Mancini, P. G. Pelicci, F. Lo Coco, C. Nervi, Cancer Res 2001, 61, 2.
[21] V. Delplace, P. Couvreur, J. Nicolas, Polymer Chemistry 2014, 5, 1529.
[22] J. Nicolas, S. Mura, D. Brambilla, N. Mackiewicz, P. Couvreur, Chem Soc Rev 2013, 42, 1147.
[23] K. M. Camacho, S. Menegatti, S. Mitragotri, Nanomedicine (Lond) 2016, 11, 1139.
[24] K. M. Camacho, S. Kumar, S. Menegatti, D. R. Vogus, A. C. Anselmo, S. Mitragotri, J Control Release 2015, 210, 198.
[25] D. R. Vogus, M. A. Evans, A. Pusuluri, A. Barajas, M. Zhang, V. Krishnan, M. Nowak, S. Menegatti, M. E. Helgeson, T. M. Squires, S. Mitragotri, J Control Release 2017, 267, 191.
[26] I. Noh, H. O. Kim, J. Choi, Y. Choi, D. K. Lee, Y. M. Huh, S. Haam, Biomaterials 2015, 53, 763.
[27] S. J. Zheng, S. P. Zheng, F. Y. Huang, C. L. Jiao, R. L. Wu, World J Gastroenterol 2007, 13, 2484.
[28] A. Pusuluri, V. Krishnan, D. Wu, C. W. t. Shields, L. W. Wang, S. Mitragotri, Bioeng Transl Med 2019, 4, e10129.
[29] T. C. Chou, Pharmacol Rev 2006, 58, 621.
[30] M. Lapteva, M. A. Sallam, A. Goyon, D. Guillarme, J. L. Veuthey, Y. N. Kalia, Expert Opin Drug Deliv 2020, 1.
[31] M. A. Sallam, M. T. Marin Bosca, J Pharm Sci 2017, 106, 3140.
[32] I. Dolz-Perez, M. A. Sallam, E. Masia, D. Morello-Bolumar, M. D. Perez Del Caz, P. Graff, D. Abdelmonsif, S. Hedtrich, V. J. Nebot, M. J. Vicent, J Control Release 2020, 318, 210.
[33] V. A. Neel, K. Todorova, J. Wang, E. Kwon, M. Kang, Q. Liu, N. Gray, S. W. Lee, A. Mandinova, J Invest Dermatol 2016, 136, 696.
[34] M. B. Brown, S. A. Jones, J Eur Acad Dermatol Venereol 2005, 19, 308.

1. A composition comprising a polymer conjugated to at least one immunomodulator.

2. (canceled)

3. The composition of claim 1, wherein the immunomodulator is a retinoid, a HDAC inhibitor, or an imidazoquinoline.

4. The composition of claim 1, wherein the immunomodulator is selected from the group consisting of: bexarotene, vorinostat, or resiquimod.

5. The composition of claim 1, wherein the composition comprises at least two immunomodulators.

6. The composition of claim 1, wherein the composition comprises at least a) a retinoid and an imidazoquinoline, b) a retinoid and a HDAC inhibitor, or c) a HDAC inhibitor and an imidazoquinoline.

7. (canceled)

8. The composition of claim 1, wherein the composition comprises resiquimod and bexarotene.

9. The composition of claim 1, wherein the composition comprises vorinostat and bexarotene.

10. The composition of claim 5, wherein the at least two immunomodulators are conjugated to the same polymer molecule such that each polymer molecule comprises at least one molecule of each of the at least two immunomodulators.

11. The composition of claim 5, wherein the at least two immunomodulators are each conjugated to separate polymer molecules such that each polymer molecule comprises only one of the at least two immunomodulators.

12.-14. (canceled)

15. The composition of claim 1, wherein the at least one immunomodulator is conjugated directly to the polymer.

16. The composition of claim 15, wherein the conjugation is via an ester bond and/or via an amide bond.

17. The composition of claim 1, wherein each polymer molecule is about 5 kDa to about 250 kDa in size.

18.-20. (canceled)

21. The composition of claim 1, wherein the polymer is a hydrophilic polymer.

22. The composition of claim 1, wherein the polymer comprises one or more of hyaluronic acid; polyglutamic acid; chitosan; dextran; and a polyvinyl alcohol.

23. The composition of claim 1, wherein the polymer comprises hyaluronic acid.

24. (canceled)

25. A cellular composition comprising a composition of claim 1, a) adhered to or located on the surface of a macrophage or monocyte, or b) present in a macrophage or monocyte.

26. (canceled)

27. (canceled)

28. A pharmaceutical composition comprising the composition of claim 1 and a pharmaceutically acceptable carrier.

29. A method of increasing the activity of level of a M1 macrophage phenotype and/or decreasing the activity or level of a M2 macrophage phenotype, the method comprising contacting at least one macrophage with a composition of claim 1.

30. A method of stimulating an immune response in a subject in need thereof, the method comprising administering a composition of claim 1.

31.-45. (canceled)

46. The composition of claim 1, wherein the polymer comprises one or more of: hyaluronic acid; polyglutamic acid; chitosan; dextran; a polyvinyl alcohol; a polymer of fumaric acid; a polymer of sebacic acid; a copolymer of fumaric acid and sebacic acid; poloxamer; polylactide; polyglycolide; polycaprolactone; copolymer of polylactic acid and polyglycolic acid; polyanhydrides; polyepsilon caprolactone; polyamide; polyurethanes; polyesteramide; polyorthoester; polydioxanone; polyacetal; polyketal; polycarbonate; polyorthocarbonate; polydihydropyran; polyphosphazene; polyhydroxybutyrate; polyhydroxyvalerate; polyalkylene oxalate; polyalkylene succinate; poly(malic acid); poly(amino acid); polyvinylpyrrolidone; polyethylene glycol; polyhydroxycellulose; polymethyl methacrylate; chitin; copolymer of polylactic acid and polyglycolic acid; poly(glycerol sebacate) (PGS); gelatin; collagen; silk; alginate; cellulose; poly-nucleic acid; cellulose acetate; cellulose diacetate; polyethylene; polypropylene; polybutylene; polyethylene terphthalate (PET); polyvinyl chloride; polystyrene; polyamide; nylon; polycarbonate; polysulfide; polysulfone; a hydrogel; an acrylic polymer; polyacrylonitrile; polyvinylacetate; cellulose acetate butyrate; nitrocellulose; copolymer of urethane and carbonate; copolymer of styrene and maleic acid; poly(ethylenimine); hyaluron; heparin; agarose; pullulan; copolymer of ethylene and vinyl (EVOH); and copolymers, terpolymers, and copolymers comprising any combinations of the foregoing.
